(19) United States
(12) Patent Application Publication
(10) Pub. No.: US 2012/0064574 A1
(43) Pub. Date: Mar. 15, 2012

(54) METHOD FOR CONVERTING LIGNOCELLULOSIC BIOMASS

(75) Inventors: Ken Tokuyasu, Ibaraki (JP); Jeung-yil Park, Ibaraki (JP); Riki Shiroma, Ibaraki (JP); Muhammad Imran Al-Haq, Ibaraki (JP)

(73) Assignee: Incorp Adm Agency, Nat'l Agr & Food Res Org, Tsukuba-shi, Ibaraki (JP)

(21) Appl. No.: 13/321,038

(22) PCT Filed: May 12, 2010
(86) PCT No.: PCT/JP2010/058011

§ 371 Date: Nov. 17, 2011

(30) Foreign Application Priority Data
May 22, 2009 (JP) 2009-123792
Sep. 25, 2009 (JP) 2009-220787

(51) Int. Cl. C12P 7/14; C07C 31/08; C12P 3/00; C08B 1/00; C01F 11/02; C12P 19/00

(52) U.S. Cl. 435/72; 423/635; 435/162; 435/168; 536/56; 568/840

(57) ABSTRACT

The present invention aims to develop a pretreatment technology for performing efficient saccharification without losing carbohydrates (in particular, free carbohydrates, starch, xylan, or the like) due to solid-liquid separation and washing steps, as a pretreatment for enzymatic saccharification of a lignocellulosic biomass feedstock (including a lignocellulosic biomass feedstock containing readily degradable carbohydrates). Provided are: a production method for a slurry to be used as a substrate for an enzymatic saccharification reaction, comprising: pulverizing an aerial part of a plant as a lignocellulosic biomass feedstock; preparing a slurry containing the biomass feedstock, calcium hydroxide, and water; subjecting the slurry to an alkali treatment; and neutralizing the slurry by introduction of and/or pressurization with carbon dioxide to decrease a pH to 5 to 7; an enzymatic saccharification method, comprising using, as a substrate, a slurry obtained by the production method for a slurry; and a production method for ethanol, comprising using, as a substrate, a saccharification product obtained by the enzymatic saccharification method.

### TECHNICAL FIELD

The present invention relates to a pretreatment technology in enzymatic saccharification of a lignocellulosic biomass feedstock, and more specifically, to a production method for a slurry to be used as a substrate for an enzymatic saccharification reaction, the method including: pulverizing an aerial part of a plant as a lignocellulosic biomass feedstock; preparing a slurry containing the biomass feedstock, calcium hydroxide, and water; subjecting the slurry to an alkali treatment; and neutralizing the slurry by introduction of and/or pressurization with carbon dioxide.

The present invention also relates to an enzymatic saccharification method using a slurry obtained by the production method as a substrate, and a production method for ethanol using carbohydrates obtained by the enzymatic saccharification method as a substrate.

### BACKGROUND ART

To meet the increasing global need for a biofuel, a competition to develop a technology for producing a bioethanol derived from carbohydrate-based biomass has emerged on a global scale. In particular, development of a technology for utilizing lignocellulosic biomass which is uncompetitive with food resources is expected to be the most important breakthrough not only in Europe and the United States but also in Japan. Development of a technology for saccharifying lignocellulosic biomass has a history of 200 years, and is conducted actively again at present. In particular, at present, there are great expectations for technologies for enzymatic saccharification mainly with cellulase instead of saccharification technologies developed mainly for saccharification with acids.

Moreover, carbohydrates in lignocellulosic biomass feedstocks are embedded in the cell walls having complex structures, and hence it is necessary to separate the carbohydrates by a pretreatment under harsh conditions before enzymatic saccharification. As pretreatment technologies for saccharification of biomass feedstocks, a steam explosion treatment with dilute sulfuric acid, a hydrothermal treatment, a caustic soda treatment, an aqueous ammonia treatment, a treatment with calcium hydroxide, and the like have hitherto been studied.

In particular, it is thought that calcium hydroxide (calcium oxide is converted into calcium hydroxide in the presence of water, and hence calcium oxide is considered to be virtually the same substance as a pretreatment reagent) is an inexpensive reagent compared with sodium hydroxide and ammonia water and has a low harmful effect, and hence it has been studied whether this reagent may be used for a pretreatment of the lignocellulosic biomass feedstock. Calcium hydroxide has a high ionization degree in an aqueous solution but has low solubility, and hence it is not highly effective to use calcium hydroxide singly for the pretreatment of woody biomass (see Non Patent Literature 1). It should be noted that, it is known that use of an oxidizing agent is effective in a treatment of the woody biomass with calcium hydroxide.

On the other hand, a plurality of articles report effectiveness of a calcium hydroxide pretreatment of a herbaceous biomass having a low lignification degree (see Non Patent Literatures 2 to 4).

It is generally thought that, in a dilute alkali treatment conducted as a pretreatment of enzymatic saccharification, esters such as an acetyl group and a feruloyl group in hemicellulose and esters in a lignin molecule are hydrolyzed, resulting in improvement of enzymatic saccharification property and solubilization of parts of lignin and silica. In this step, part of hemicellulose is also released and solubilized, but most of cellulose and hemicellulose remain as solid matter in the biomass, and hence the subsequent enzymatic saccharification can be conducted efficiently.

However, such dilute alkali treatment step requires a solid-liquid separation step for separating reagents such as an acid and an alkali or water-soluble components from solid matter derived from the cell wall and washing and neutralization steps before a saccharification step using an enzyme such as cellulase which acts under mildly acidic conditions. Even in the case of the hydrothermal treatment, it is desired to conduct washing to remove over-degradation products or free lignin.

Meanwhile, in the calcium hydroxide pretreatment step, a mixture mainly containing a crushed/pulverized product of the biomass feedstock, calcium hydroxide, and water is allowed to react at room temperature or with heating to exert a dilute alkali treatment effect. However, cations (such as Na⁺, Ca²⁺, and Mg²⁺) in the alkali are bonded strongly to the biomass (mainly carboxyl groups in hemicellulose and phenol groups in lignin) in the pretreatment reaction and cannot be removed completely by simple water washing. Further, the cations released from the biomass show alkalinity, and hence the washing requires a large amount of water (see Non Patent Literature 5).

As a neutralization method for the alkali-pretreated product, there have been studied a method involving neutralization by washing with water (see Non Patent Literature 6), a method involving washing with water after neutralization with hydrochloric acid (see Non Patent Literature 4), a method involving washing with water after neutralization with acetic acid (see Non Patent Literature 7), a method involving washing with water after neutralization with citric acid (see Non Patent Literature 8), a method involving the above-mentioned neutralization methods in combination (see Non Patent Literature 2), and the like.

However, the neutralization methods listed above may cause a loss of solid matter derived from the cell wall and soluble carbohydrates in the solid-liquid separation step or the washing step, resulting in a decrease in the yield of the carbohydrates.

In addition, specific drawbacks of hydrochloric acid, sulfuric acid, and washing with water as particularly general methods among the above-mentioned methods are shown below.

(1) Hydrochloric acid: After neutralization, water-soluble calcium chloride is produced. A process for the neutralization is simple, but it is difficult to recycle calcium chloride, and it highly costs for the acid and for both maintenance and operation of the washing step. In addition, in order to decrease the ion concentration before the saccharification step, processes of the solid-liquid separation and washing are required, and the processes are conducted using a large amount of water, resulting in discharging a waste liquid and losing fibrous solidmatter and free carbohydrates. The treatment of the waste liquid becomes difficult by calcium chloride generated in the neutralization process and solubilized lignin and xylan having a reduced molecular weight generated in the alkali pretreatment. Further, in order to conduct a saccharification enzyme reaction continuously after neutralization and washing, it is necessary to further adjust the pH in a reactor, and hence there are risks of an increase in reagent cost and microbial contamination in the washing step.

(2) Sulfuric acid: After neutralization, insoluble gypsum is precipitated. The generated gypsum has extremely poor solubility and hardly causes inhibition by salts in an enzymatic reaction and fermentation with microorganisms. A process for the neutralization is simple, but it is difficult to recycle reagents, and it highly costs for a treatment of the gypsum, for sulfuric acid, and for both maintenance and operation of the washing step. In addition, in order to reduce the concentration of solid matter in saccharification, a process for separating powdery gypsum from fibrous solid matter should be conducted, and the process requires a large amount of water, resulting in a discharge of a waste liquid and losing fibrous solid matter and free carbohydrates. In the case where a biomass treated has a small particle size, it is difficult to separate the gypsum generated in neutralization process from the biomass after the treatment, and as is the case with neutralization with hydrochloric acid, the treatment of the waste liquid becomes difficult by solubilized lignin and xylan having a reduced molecular weight generated in the alkali pretreatment. Further, in order to conduct the saccharification enzyme reaction continuously after neutralization and washing, it is necessary to further adjust the pH in a reactor, and hence there are risks of an increase in reagent cost and microbial contamination in the washing step.

(3) Washing with water: an interaction between calcium hydroxide and fibrous solid matter lowers the rate of a decrease in the pH, and hence the efficiency of the washing step becomes very low, resulting in the generation of a large amount of waste water. The fibrous solid matter and free carbohydrates are lost by washing. The solubilized lignin and silica as well as xylan having a reduced molecular weight generated in the alkali pretreatment are not reprecipitated in the water-washing step and discharged in the waster liquid in large amounts compared with neutralization with hydrochloric acid or sulfuric acid, which makes the treatment of the waste liquid more difficult. Further, in order to conduct the saccharification enzyme reaction continuously after neutralization and washing, it is necessary to further adjust the pH in a reactor, and hence there are risks of an increase in reagent cost and microbial contamination in the washing step.

As described above, the conventional neutralization methods require the solid-liquid separation and washing steps, and in particular, in the case where saccharification is conducted using rice straw containing readily degradable carbohydrates such as sucrose and starch as biomass feedstocks, there is a risk of a loss of sucrose and starch due to solid-liquid separation and washing and neutralization after a chemical pretreatment for improving saccharification property of cellulose.

Further, the solid-liquid separation step is conducted using a centrifuge, a screen-type separation device, or the like, and hence there is a problem of an increase in cost due to introduction and operation of the separation device. The washing and neutralization steps require introduction of a continuous washing device and use of a large amount of water, and hence a cost for treating a waste liquid increases.

Therefore, there has been required development of a technology for efficient saccharification by improving the solid-liquid separation step and washing and neutralization steps after the pretreatment to prevent a loss of solid matter derived from the cell wall and free carbohydrates (technology capable of drastically reducing a starting material cost, a reagent cost, and facility and operation costs).

### CITATION LIST

### Non Patent Literature

[Non Patent Literature 1] Vincent S. Chang, Murlidhar Nagwani, Chul-Ho Kim, and Mark T. Holtzapple, Applied Biochemistry and Biotechnology, 2001, 94, 1
[Non Patent Literature 2] Vincent S. Chang, Barry Burr, and Mark T. Holtzapple, Applied Biochemistry and Biotechnology, 1997, 63-65, 3
[Non Patent Literature 3] Vincent S. Chang, Murlidhar Nagwani, and Mark T. Holtzapple, Applied Biochemistry and Biotechnology, 1998, 74, 135
[Non Patent Literature 4] Sarita C. Rabelo, Rubens M. Filho, and Aline C. Costa, Applied Biochemistry and Biotechnology, 2008, 153(1-3), 139
[Non Patent Literature 5] Washing, Screening and Bleaching of Pulp, Japan Technical Association of the Pulp and Paper Industry, 2000
[Non Patent Literature 6] Sarita C. Rabelo, Rubens Maciel Filho, and Aline C. Costa, Applied Biochemistry and Biotechnology 2008, 148, 45
[Non Patent Literature 7] William E. Karr, Mark T. Holtzapple, Biotechnology and Bioengineering, 1998, 59(4), 419
[Non Patent Literature 8] William E. Karr, Mark T. Holtzapple, Biomass and Bioenergy, 2000, 18, 189

### SUMMARY OF INVENTION

### Technical Problem

In order to solve the above-mentioned problems, an object of the present invention is to develop a pretreatment technology for performing efficient saccharification without losing carbohydrates (in particular, free carbohydrates, starch, xylan, or the like) due to solid-liquid separation and washing steps, as a pretreatment for enzymatic saccharification of a lignocellulosic biomass feedstock (including a lignocellulosic biomass feedstock containing readily degradable carbohydrates).

### Solution to Problem

The inventors of the present invention have made intensive studies in order to solve the above-mentioned conventional problems, and as a result, have focused on carbon dioxide as an acid used for neutralization when an alkali treatment is conducted with calcium hydroxide as a pretreatment for enzymatic saccharification of a lignocellulosic biomass feedstock. As a result, the inventors have found that: in neutralization with carbon dioxide, calcium carbonate, which is generated by the neutralization, has very poor solubility and hardly causes inhibition by salts in an enzymatic reaction and fermentation with microorganisms even if calcium carbonate remains in a reaction system; and that the neutralization can be conducted relatively easily at the lowest cost; and that calcium carbonate can be regenerated into calcium oxide by thermal decomposition.

Therefore, the inventors of the present invention have found that an enzymatic saccharification reaction and ethanol fermentation can be conducted directly ‘without solid-liquid separation and washing’ by pulverizing the lignocellulosic biomass feedstock, conducting the alkali treatment with calcium hydroxide, and neutralizing the resultant by introduction of and/or pressurization with carbon dioxide to prepare a slurry, and have completed the present invention.

That is, a first aspect of the present invention relates to a production method for a slurry to be used as a substrate for an enzymatic saccharification reaction, comprising: pulverizing an aerial part of a plant as a lignocellulosic biomass feedstock; preparing a slurry containing the biomass feedstock, calcium hydroxide, and water; subjecting the slurry to an alkali treatment; and neutralizing the slurry by introduction of and/or pressurization with carbon dioxide to decrease a pH to 5 to 7.

A second aspect of the present invention relates to a production method for a slurry according to the first aspect, in which the alkali treatment is conducted at 80 to 180° C. for 10 minutes to 3 hours.

A third aspect of the present invention relates to a production method for a slurry according to the first aspect, in which the alkali treatment is conducted at 0° C. to 50° C. for 3 days or more.

A fourth aspect of the present invention relates to a production method for a slurry according to any one of the first to third aspects, further comprising the step of grinding solid matter in the slurry before or after the neutralization.

A fifth aspect of the present invention relates to a production method for a slurry according to any one of the first to fourth aspects, in which the aerial part of the plant includes one or more selected from rice, wheat, barley, corn, sugarcane, sorghum, erianthus, pasture plants, and monocotyledonous weeds.

A sixth aspect of the present invention relates to a production method for a slurry according to any one of the first to fifth aspects, in which the aerial part of the plant is a non-edible part.

A seventh aspect of the present invention relates to an enzymatic saccharification method comprising: adding a saccharification enzyme for at least one kind of starch, β-(1→3), (1→4)-glucan, cellulose, xylan, and partial degradation products thereof to a slurry obtained by the production method according to any one of the first to sixth aspects; and conducting an enzymatic saccharification reaction with, if necessary, introduction of and/or pressurization with carbon dioxide to prevent an increase in pH.

An eighth aspect of the present invention relates to a production method for ethanol, comprising: adding microorganisms for ethanol fermentation to a slurry containing a saccharification product obtained by the enzymatic saccharification method according to the seventh aspect; and conducting ethanol fermentation with, if necessary, introduction of and/or pressurization with carbon dioxide to prevent an increase in pH.

A ninth aspect of the present invention relates to a production method for ethanol, comprising, in the enzymatic saccharification reaction according to the seventh aspect, further adding microorganisms for ethanol fermentation in addition to the saccharification enzyme to conduct the enzymatic saccharification reaction and ethanol fermentation as simultaneous saccharification and fermentation.

A tenth aspect of the present invention relates to a production method for ethanol according to the eight or ninth aspect, in which the microorganism for ethanol fermentation is yeast.

An eleventh aspect of the present invention relates to a bioethanol, which is obtained by the method according to any one of the eight to tenth aspects.

A twelfth aspect of the present invention relates to a collection method for an inorganic material containing calcium salts, the method comprising: conducting the enzymatic saccharification reaction according to the seventh aspect; collecting a saccharification product; conducting solid-liquid separation of a residue by membrane filtration or centrifugation; and combusting the resultant solid matter to collect ash.

A thirteenth aspect of the present invention relates to a collection method for an inorganic material containing calcium salts, the method comprising: conducting the ethanol fermentation according to any one of the eighth to tenth aspects; collecting ethanol; conducting solid-liquid separation of a residue by membrane filtration or centrifugation; and combusting the resultant solid matter to collect ash.

A fourteenth aspect of the present invention relates to a collection method for an inorganic material containing calcium salts according to the twelfth or thirteenth aspect, in which the inorganic material containing calcium salts includes a phosphate.

A fifteenth aspect of the present invention relates to an inorganic material containing calcium salts, which is obtained by the method according to any one of the twelfth to fourteenth aspects.

### Advantageous Effects of Invention

According to the present invention, it is possible to directly conduct a saccharification reaction and ethanol fermentation ‘without conducting solid-liquid separation and washing steps’ because a pH suitable for enzymatic saccharification and fermentation can be maintained stably without discharging solid matter derived from the cell wall and free carbohydrates in a reaction container. That is, it is possible to simultaneously conduct a series of steps of pretreatment, saccharification, and ethanol fermentation in one reactor.

Thus, according to the present invention, it is possible to provide a pretreatment technology for performing efficient saccharification without losing carbohydrates (in particular, free carbohydrates) due to solid-liquid separation and washing steps, as a pretreatment for enzymatic saccharification of a lignocellulosic biomass feedstock (in particular, a lignocellulosic biomass feedstock containing readily degradable carbohydrates).

Further, according to the present invention, it is possible to conduct the treatment with calcium hydroxide and saccharification reaction using, as biomass feedstocks, not only biomass feedstock including only fibers (cellulose and hemicellulose) but also stem and leaf parts and whole aerial parts of plants of rice straw, sugarcane, and the like containing readily degradable carbohydrates such as starch and sugar among the lignocellulosic biomass feedstock, thereby collecting carbohydrates efficiently from both the readily degradable carbohydrates and cellulose and hemicellulose, and the carbohydrates can be used in ethanol fermentation step.

That is, according to the present invention, it is possible to produce the ‘bioethanol’ efficiently from the lignocellulosic biomass feedstock.

### BRIEF DESCRIPTION OF DRAWINGS

FIG. 1 is a graph showing glucan saccharification rates and xylan saccharification rates at various pH values in Test Example 1.

FIG. 2 is a graph showing a pH variation in neutralization of a calcium hydroxide suspension with carbon dioxide in Test Example 2.

FIG. 3 is a graph showing a pH variation of a rice straw slurry neutralized with carbon dioxide after a treatment with calcium hydroxide in Example 1.

FIG. 4 is a schematic view illustrating a vial bottle in Example 2.

FIG. 5 is a graph showing a temporal change in an ethanol conversion rate in simultaneous saccharification and fermentation in Example 11.

FIG. 6 is a graph showing temporal changes in amounts of free glucose and xylose in a fermenter in Example 11.

### DESCRIPTION OF EMBODIMENTS

The present invention relates to a pretreatment technology in enzymatic saccharification of a lignocellulosic biomass feedstock, and more specifically, to a production method for a slurry to be used as a substrate for an enzymatic saccharification reaction, the method comprising: pulverizing an aerial part of a plant as a lignocellulosic biomass feedstock; preparing a slurry containing the biomass feedstock, calcium hydroxide, and water; subjecting the slurry to an alkali treatment; and neutralizing the resultant by introduction of and/or pressurization with carbon dioxide to prepare a slurry.

[Biomass Feedstock]

As the “lignocellulosic biomass feedstock” as targets of the present invention, aerial parts of plants may be used.

The materials are broadly classified into woody feedstocks and herbaceous feedstocks. In addition to the materials, seaweeds, waterweeds, and the like may be used as the target biomass feedstocks of the present invention as ones similar to the lignocellulosic biomass feedstock.

Examples of the woody feedstocks include stems, branches, leaves, and nuts of needle leaf trees, broadleaf trees, and gymnosperms. However, in general, the herbaceous (biomass) feedstocks have a lignification degree lower than that of the woody (biomass) feedstocks, and hence pretreatment conditions can be adjusted to bemild. Therefore, the herbaceous (biomass) feedstocks are preferably used as the biomass feedstock of the present invention.

As the herbaceous feedstocks, there may be used whole aerial parts of rice, wheat, barley, corn, sugarcane, sorghum, erianthus, pasture plants, and monocotyledonous weeds.

Further, a non-edible part is desirably used as the lignocellulosic biomass feedstock of the present invention in order to avoid competition with food production.

Specific examples thereof include corn stems and leaves (corn stover) accumulated in agricultural fields in production of corn ethanol; bagasse obtained after extraction of sugarcane juice; rice straw, wheat straw, barley straw, and chaffs produced as by-products in production of main crops; sweet sorghum and erianthus produced as the so-called resource crops; pasture plants; and whole aerial parts of rice plants.

The lignocellulosic biomass feedstock includes ones containing readily degradable carbohydrates. Of those, in particular, for the rice straw and sugarcane bagasse, development of a pretreatment technology for improving saccharification property of cellulose and hemicellulose while collecting readily degradable carbohydrates such as starch and sucrose has been required, and the present invention solves the problem.

In the present invention, the above-mentioned biomass feedstocks are pulverized before use.

The optimum pulverization degrees of the biomass feedstock in the present invention vary depending on the shapes, water contents, or pulverization characteristics of the biomass feedstocks.

For example, in the case where a slurry is prepared using rice straw as a sample, the effect of the alkali treatment can be provided even for long rice straw after thrashing or rice straw cut to sizes of about a few centimeters. However, in the case of a sample pulverized so as to have an average particle size ranging from about several millimeters to a few hundreds of micrometers or less, permeability of a chemical solution and a surface area of a substrate are improved, resulting in an increase in the saccharification efficiency after the pretreatment.

Although the reaction efficiency is probably improved as the biomass feedstock is pulverized more finely unless a wear damage of the biomass feedstock or coating of the substrate are caused by heat in pulverization, optimization is required in view of saccharification efficiency, pulverization cost, and handling property depending on the biomass feedstock. For example, the alkali treatment is expected to soften the biomass and to decrease a mechanical strength, resulting in an increase in the energy efficiency of the subsequent pulverization step.

In the present invention, it is not necessary to conduct separation of salts from the pretreated biomass feedstock (solid-liquid separation or washing) after neutralization, and hence even if the particle size of the sample is a few hundreds of micrometers or less, the sample is not lost and deterioration of the handling property hardly occurs. This is a great advantage of the present invention. The efficiency of the alkali treatment is expected to be improved by a method involving allowing an alkali liquid to permeate a pulverized biomass feedstock while grinding the material using, for example, a grinder for grinding using a grindstone or the like.

[Alkali Treatment]

In the present invention, the biomass feedstock is pulverized, and a slurry containing the biomass feedstock, calcium hydroxide, and water is prepared and subjected to an alkali treatment.

In the alkali treatment, a reaction mixture is prepared by various methods including: a method involving adding water to a biomass and then mixing calcium hydroxide or a water suspension thereof; inversely, a method involving adding calcium hydroxide powder and then adding water or water vapor; a method involving adding calcium hydroxide in multiple steps; and a method involving adding and mixing only calcium hydroxide by use of water in a biomass. Meanwhile, in order to improve permeability of water or a reagent to the biomass feedstock, a method involving adding a surfactant, a method involving removing air bubbles under reduced pressure, a method involving promoting permeation of a liquid by reducing air bubbles under pressure, and the like may be employed.

It is generally thought that esters such as an acetyl group and a feruloyl group in hemicellulose and esters in a lignin molecule are hydrolyzed by the alkali treatment, resulting in both improvement of enzymatic saccharification property and solubilization of parts of lignin and silica. In the alkali treatment, part of hemicellulose is also released and solubilized, but most of cellulose and hemicellulose remain as solid matter in the biomass, and serve as substrates of the subsequent enzymatic saccharification.

In the present invention, the alkali treatment is conducted using ‘calcium hydroxide (or calcium oxide)’. Use of another alkali such as sodium hydroxide, potassium hydroxide, magnesium hydroxide, or ammonia water is inappropriate in terms of an effect of decreasing the pH of a biomass powder slurry, in terms of difficulty in producing precipitates of a salt which causes inhibition of an enzymatic reaction or fermentation in neutralization with carbon hydroxide, or in terms of reagent collection or a reagent cost.

It should be noted that the addition ratio of calcium hydroxide used in the treatment may be 2 to 80%, desirably 10 to 40% per dry weight of the biomass feedstock.

In such case, the water content in the pretreatment reaction system may be adjusted to 1 to 40 times, desirably 3 to 20 times that of the biomass feedstock. Moreover, water in the biomass feedstock may be included in the water content. Further, the amount of water added may be lowered by increasing the pulverization degree of the biomass feedstock.

The treatment with calcium hydroxide may be conducted at a high temperature of 80° C. or more or at ambient temperature or around room temperature.

High Temperature Condition

In the case where the treatment with calcium hydroxide is conducted under a high temperature condition, the treatment can be effectively conducted at a temperature of 80° C. or more, desirably about 100° C. or more for several hours. It should be noted that, if the temperature exceeds 180° C., phenomena of an increase in cost for the heat treatment and a decrease in sugar yield are observed. Therefore, in the present invention, the treatment is conducted under a condition of 80 to 180° C., more desirably 80° C. to 160° C.

The treatment time should be about 10 minutes or more required for heat transfer, and the time ranges desirably from about 10 minutes to 3 hours, preferably from about 30 minutes to 2 hours. In addition, in the case where the slurry is prepared using water vapor, a water addition treatment and a heat treatment may be conducted simultaneously.

Ambient Temperature or Room Temperature Condition

In the case where the treatment with calcium hydroxide is conducted under an ambient temperature or room temperature condition, it is effective to preserve the sample specifically at 0° C. to 50° C., desirably at 10° C. to 40° C. which is about room temperature for 3 hours or more, desirably for 3 days or more, more desirably for 6 days or more. Further, the ambient temperature often falls below freezing in winter, and the present invention includes a case where preservation is conducted at ambient temperature under such conditions.

It should be noted that, in the case of the alkali treatment at ambient temperature or room temperature, not only a pretreatment effect under alkali conditions but also a ‘preservation effect’ are expected. Therefore, when preservation is conducted for about 3 hours to a few hundreds of days or more, long-term storage and use of crops can be achieved. In particular, a biomass feedstock such as a rice straw or sugarcane pulverized product having a high water content can be stored without drying, and hence the present invention is important as a technology for reducing a drying cost and for suppressing changes in the characteristics of the biomass feedstock due to drying, for example. As a method of preserving a biomass feedstock such as rice straw without drying, inoculation of a lactic acid bacterium, inoculation of ammonia, inoculation of urea, and the like have hitherto been known. However, the lactic acid bacterium has problems such as consumption of parts of carbohydrates in lactic acid fermentation, inhibition of ethanol fermentation by lactic acid, and contamination during ethanol fermentation by yeast cells with the lactic acid bacterium. Further, ammonia has drawbacks in that it is relatively expensive and has odor and toxicity to lower working efficiency. Urea is expected to have practicality in silage production, but in the case where urea is used only as an ethanol fermentation substrate, it is feared that harmful substances are produced. Based on those standpoints, a non-drying preservation method using calcium hydroxide is very effective and suitable for practical use and further exerts effectiveness in the technology of the present invention. In particular, starch and sucrose contained in the biomass feedstock such as the rice straw or sugarcane pulverized product are present almost stably in an alkali and hence can be maintained while avoiding deterioration due to microbial contamination and plant metabolism. Further, the method has a high effect as a pretreatment, and hence a cost for heating in the pretreatment can be drastically reduced compared with a pretreatment conducted at a high temperature.

In addition, in order to promote degradation of lignin and to appropriately prevent a decrease in sugar yield due to β-elimination, it is effective to add an oxidant such as anthraquinone or molecular oxygen. Further, when the solid matter in the slurry after the alkali treatment is ground before neutralization with carbon dioxide, the enzymatic reaction in the subsequent step can be promoted.

[Neutralization with Carbon Dioxide]

In the present invention, the solution after the treatment with calcium hydroxide (alkali treatment) is neutralized by introduction of and/or pressurization with carbon dioxide to decrease the pH.

The pH after neutralization is desirably adjusted to 5 to 7, preferably to be mildly acidic, i.e., 6.5 or less where many of saccharification enzymes have high activities. Specifically, the pH is desirably adjusted to 5 to 6.5.

Specific examples of the neutralization with carbon dioxide include: a method involving directly introducing carbon dioxide (through bubbling, addition of carbonated water, blowing from the upper side, or the like) into the solution after the alkali treatment; and a method involving pressurizing the solution (to a positive pressure) with carbon dioxide using a closed container. In addition, carbon dioxide can be more efficiently dissolved by stirring, shaking, a low-temperature or high-pressure treatment, or the like. Further, the methods may be employed in combination.

It should be noted that, in the present invention, carbon dioxide discharged from the reaction system may be collected using an unclosed container by a downward substitution method or the like, but use of the closed container is desirable from an economical standpoint.

Carbon dioxide pressurization can suppress a gradual pH increase to adjust the pH to a constant level in the above-mentioned predetermined range. Further, when the pressure in the container gradually decreases due to consumption of carbon dioxide in the positive pressure container, fresh carbon dioxide can be automatically introduced by using a pressure indicator switch or the like.

A source of the carbon dioxide gas used in the present invention may be commercially available carbon dioxide, gas after boiler combustion, gas generated in fermentation, or the like. It is generally thought that the need for purification of the gas is not high.

Further, the step of producing ethanol from a lignocellulosic biomass feedstock includes a step of combusting a residue after saccharification and fermentation of lignin or the like and a step of fermenting ethanol, and hence the gas can be available from a conversion factory. Moreover, in the case where a large-scale factory for production of a bioethanol from sucrose or starch or a factory for conducting a boiler combustion step is adjacent, carbon dioxide is expected to be supplied more efficiently. A neutralization system using calcium hydroxide-carbon dioxide can promote precipitation of a substance such as free lignin and reduce a cost for a waste liquid treatment by the so-called overliming effect.

It should be noted that, carbon dioxide is further generated from a reaction solution in ethanol fermentation which is a step described later, and carbon dioxide released from the reaction solution may be stored and used.

Subsequent to neutralization of the slurry after the alkali treatment with carbon dioxide to maintain the pH to the above-mentioned predetermined range, an enzyme may be ‘directly’ added to the slurry to conduct the saccharification reaction. Therefore, in the present invention, it is possible to completely omit a step that may cause loss of carbohydrates (in particular, readily degradable carbohydrates) such as solid-liquid separation or washing after the pretreatment.

In addition, the slurry after the neutralization with carbon dioxide has a pH value suitable for the activity of the saccharification enzyme, and calcium is precipitated as a salt. Most of calcium carbonate is converted into solid matter and is not present as a solute, and hence the effect of the salt on the enzymatic activity is estimated to be very small.

Further, many of calcium carbonate crystals generated after neutralization are present in contact with a pretreated biomass, and hence when the pretreated product is subjected to a wet-milling treatment before saccharification, the calcium carbonate crystals are expected to play a role as an abrasive. Before the enzymatic saccharification reaction, or at the time from addition of the enzyme to the enzymatic saccharification, if the solid matter in the slurry after neutralization with carbon dioxide is ground, the saccharification efficiency may increase.

In the present invention, even if unreacted calcium hydroxide is present in a minute amount, the effect on enzyme stability can be suppressed to a minimum level by rapid neutralization under a carbon dioxide gas atmosphere.

[Enzymatic Saccharification Reaction]

Major polysaccharides in the lignocellulosic biomass feedstock (in particular, herbaceous (biomass) feedstocks) to be used as the biomass feedstock in the present invention include starch, β-(1→3), (1→4)-glucan, cellulose, and xylan. In the present invention, an enzyme having an activity to saccharify at least one kind of the polysaccharides or partial degradation products thereof (in addition, enzyme having an activity to promote saccharification) is added.

It should be noted that, preferably, two or more kinds of enzymes are desirably added in combination so as to saccharify all the polysaccharides or partial degradation products thereof.

As the saccharification enzyme, there may be used a cellulase preparation, a hemicellulase preparation, and a β-glucosidase preparation. Specific examples thereof include α-amylases, β-amylases, glucoamylases, pullulanases, isoamylases, α-glucosidases, lichenases, cellobiohydrolases, endoglucanases, β-glucosidases, cellobiose dehydrogenases, xylanases, α-L-arabinofuranosidases, β-D-xylosidases, α-glucuronidases, β-glucuronidases, acetylxylanesterases, feruloylesterases, β-mannanases, β-D-mannosidases, α-galactosidases, β-galactosidases, xyloglucanases, galactanases, arabinanases, pectinases, pectin methyl esterases, and pectin acetyl esterases.

Many of enzymes capable of hydrolyzing cell wall components such as cellulase and hemicellulase included in the above-mentioned saccharification enzymes have high activities at about pH 4.5 to 5.5, and many of the enzymes maintain high activities even at about pH 6.5.

In the present invention, the saccharification reaction is conducted while preventing an increase in pH (under a constant pH condition) by using carbon dioxide when needed in the saccharification reaction.

It should be noted that, if a saccharification enzyme having a lowered activity at about pH 6.5 is highly stable, the enzyme may be employed at a usual dosage or at an increased dosage.

Meanwhile, in the case of an enzyme having low stability, an enzymatic activity can be optimized by adjusting the dosage so that a sufficient catalyst activity can be achieved until the enzyme is inactivated.

Further, as described above, although many of enzyme preparations for saccharifying biomasses can be used at about pH 6.5, an ‘enzyme for saccharification having a particularly high activity’ which is active at about pH 6.5 and is obtained by screening from nature, a mutant enzyme obtained by modifying a protein structure so as to have improved catalytic properties and stability, or the like may be used in the saccharification step. For example, an enzyme derived from a filamentous fungus belonging to the genus Humicola, in particular, Humicola insolens may be used as β-glucosidase having a high activity at about pH 6.5.

Although the saccharification reaction may be conducted at a temperature suitable for the activity of the saccharification enzyme, an enzyme having high heat resistance may be added sequentially with a decrease in the temperature of the pretreatment product (slurry neutralized with carbon dioxide after the alkali treatment) in heating in the alkali treatment to improve the efficiency of the saccharification step.

For example, in the case where the temperature decreases to about 70° C. to 110° C. at which starch gelatinization is liable to occur, the efficiency of liquefaction of starch is improved by conducting the saccharification reaction with heat-resistant amylase being added thereto.

Further, many of cellulase preparations and hemicellulase preparations in commercially available enzyme preparations act stably at about 50° C., and hence an enzyme is desirably added when the temperature of the pretreatment product decreases to about 50° C.

It should be noted that, saccharification may be conducted with not only the enzyme (including a functional protein) but also a factor capable of promoting the enzymatic saccharification reaction such as a surfactant being added.

Examples of the saccharification product obtained after the enzymatic saccharification reaction include glucose, xylose, arabinose, galactose, mannose, rhamnose, fructose, glucuronicacid, and galacturonic acid. In particular, main ethanol fermentation substances include glucose, xylose, galactose, and fructose.

[Ethanol Fermentation]

In the present invention, ethanol fermentation is conducted by adding microorganisms for ethanol fermentation to a slurry containing a saccharification product obtained by the above-mentioned enzymatic saccharification reaction and optionally using carbon dioxide to prevent an increase in pH (under a constant pH condition).

It should be noted that, the ethanol fermentation is conducted using not only the saccharification product obtained by the above-mentioned enzymatic saccharification reaction but also carbohydrates per se contained in the biomass feedstock (such as intrinsic glucose, fructose, and sucrose) as substrates.

The slurry in the present invention hardly causes inhibition in normal ethanol fermentation as well as in the above-mentioned enzymatic reaction, and hence microorganisms for ethanol fermentation may be ‘directly’ added to the slurry to conduct the ethanol fermentation. Therefore, in the present invention, it is possible to completely omit a step that may cause loss of carbohydrates such as solid-liquid separation or washing before the fermentation. That is, the ‘bioethanol’ can be produced efficiently.

In addition, the slurry has a pH value suitable for the ethanol fermentation, and calcium is precipitated as a salt. Most of calcium carbonate is converted into solid matter and is not present as a solute, and hence the effect of the salt on the ethanol fermentation is estimated to be very small.

Moreover, according to the present invention, the enzymatic saccharification reaction and the ethanol fermentation may be conducted as ‘simultaneous saccharification and fermentation’ by further adding microorganisms for ethanol fermentation to the slurry after neutralization with carbon dioxide before the enzymatic saccharification reaction together with the above-mentioned saccharification enzyme.

The simultaneous saccharification and fermentation to simultaneously conduct saccharification and fermentation can shorten the time for obtaining ethanol which is a fermentation product and can reduce a facility cost. Further, in a consolidated bioprocess developed by sophisticating the simultaneous saccharification and fermentation step, the neutralized slurry in the present invention may be used as a substrate.

In addition, a decrease in pH in a fermenter due to an organic acid produced as a by-product in the ethanol fermentation may cause inhibition of the ethanol fermentation or inhibition of growth of the microorganism. However, in the ethanol fermentation in the present invention, the organic acid generated in the fermentation is naturally neutralized by calcium carbonate generated in the process of neutralization with carbon dioxide, and hence a cost of an additional reagent for controlling the pH in the fermenter can be reduced.

As the microorganisms for ethanol fermentation to be used in the present invention, there may be used ethanol-producing microorganisms including: yeasts such as Saccharomyces cerevisiae, Pichia stipitis, Candida shehatae, and Kluyveromyces marxianus; ethanol-producing basidiomycetes and ascomycetes; and bacteria such as Zymomonas mobilis.

It should be noted that, at the time of fermentation, carbon dioxide generated, carbon dioxide introduced for maintaining the pH, or the like keeps the pH in the reaction solution around 6.5 or less. The pH of around 6.5 falls within a pH range in which many of yeasts, bacteria, and filamentous fungi can be grown, and hence various genetically-modified microorganisms such as Escherichia coli, Saccharomyces cerevisiae, and Corynebacterium sp. may be used.

Meanwhile, a rate of conversion of the biomass feedstock into ethanol can be improved by adding a plurality of microorganisms (for example, microorganism having a fermentation ability for glucose or sucrose and microorganism having a fermentation ability for xylose) simultaneously or one by one with time to conduct fermentation.

It should be noted that, the technology can be applied not only in the ethanol fermentation but also in various biorefinery steps by modifying types of fermenting microorganisms or culture conditions.

[Collection of Inorganic Material]

In the present invention, an inorganic material containing calcium salts (ash) can be collected by: conducting the above-mentioned enzymatic saccharification reaction or ethanol fermentation; collecting a target substance; conducting solid-liquid separation of a residue by membrane filtration or centrifugation; and combusting the resultant solid matter (solid matter containing calcium carbonate, lignin, or a fermenting microorganism). Further, during the above-mentioned procedures, heat derived from lignin may be collected.

The present invention has a merit in that combustion of lignin and collection of the inorganic material containing calcium salts can be achieved by conducting the step of combusting the solid matter once.

The collected inorganic material containing calcium salts (ash) can be used as calcium oxide and can be reused in the calcium hydroxide pretreatment in the present invention.

In addition, the ash contains an inorganic component derived from the biomass feedstock, such as silica obtained from the rice straw, and in the case of using the ash as a material for rice cultivation, the ash containing silica has an added value as a fertilizer.

Collection and reuse of inorganic nutrients in a biomass conversion process are very important. There has been required development of a technology for collecting phosphorus derived from a biomass feedstock, a fermenting microorganism, or any other biogenic substance or phosphorus contained in a reagent such as an enzyme preparation and reusing it as a plant nutrient source. In the present invention, the inventors have focused on a phenomenon in which phosphoric acid is bonded to a calcium ion to form various poorly-soluble salts, and have invented a method involving collecting phosphorus as the ash by combusting a distilled residue containing calcium.

As described above, the ash after combustion contains calcium and other inorganic metals having added values and is expected to give an inorganic salt material having characteristics reflecting the biomass feedstock and conversion steps. The composition of the ash varies depending on the combustion temperature. In particular, calcium carbonate is changed efficiently into calcium oxide at 820° C. or more, particularly at about 1,000° C. to 1,100° C. In the case where adjustment of the alkali content is important while retaining calcium carbonate as a by-product, a change in the composition can be controlled by changing temperature conditions. The resultant ash can be used not only as an agriculture-related material such as a fertilizer or a soil conditioner but also as a pavement material, a metal collection material, and a material such as a calcium hydroxide source in overliming or the like.

### EXAMPLES

Hereinafter, the present invention is described in detail by way of examples and the like. However, the scope of the present invention is not limited thereto.

### Preparation Example 1

### Preparation of Lignocellulosic Biomass Feedstock

In the following experiment examples and examples, the lignocellulosic biomass feedstock used as the biomass feedstock includes rice straw (variety name: Koshihikari, Leaf Star), barley straw (variety name: Silky Snow), sugarcane bagasse (available from a sugar factory in Japan), sorghum bagasse (variety name: SIL-05), and sugarcane (variety name: Nif8).

Each biomass feedstock was prepared as a powder by drying the material at 65° C. so as to have a water content of 5% or less and pulverizing the dried material so as to have a particle size of 1 mm or less.

### Measurement Example 1

### (1) Contents and Saccharification Rates of Various Carbohydrates

### A. Measurement of Glucose Content and Xylose Content

The lignocellulosic biomass powder (rice straw, sugarcane, barley straw, sorghum, or sugarcane bagasse) or the powder after the alkali treatment was weighed in an amount of 100 mg and subjected to a two-step sulfuric acid treatment (treated with 1 mL of 72% sulfuric acid at 30° C. for 1 hour, then diluted 8-fold with water, and treated at 100° C. for 2 hours). Further, part of the treated product was sampled and neutralized with a 10% NaOH aqueous solution.

Then, the glucose content per dry weight was measured using a Glucose C-II Test Wako (Wako Pure Chemical Industries, Ltd.). Further, the xylose content per dry weight was measured using a D-xylose kit (Megazyme).

### B. Calculation of Glucan Content and Xylan Content

The glucan content and the xylan content in the biomass powder before and after the alkali treatment were calculated by Equations 1 and 2 below.

Glucan content(%)=100×(glucose amount×0.90)/dry weight of biomass feedstock [Eq. 1]

Xylan content(%)=100×(xylose amount×0.88)/dry weight of biomass feedstock [Eq. 2]

C. Calculation of Glucan Saccharification Rate and Xylan Saccharification Rate after Saccharification Reaction

The glucan saccharification rate and the xylan saccharification rate after each saccharification reaction were calculated by Equations 3 and 4 below.

Glucan saccharification rate (%)=100×(enzymatic saccharification glucose amount×0.90)/glucan content in biomass feedstock [Eq. 3]

Xylan saccharification rate (%)=100×(enzymatic saccharification xylose amount×0.88)/xylan content in biomass feedstock [Eq. 4]

D. Calculation of Saccharified Glucan Yield and Saccharified Xylan Yield after Saccharification Reaction

In the case where neutralization and washing steps of a sample are required after the alkali treatment, a loss of a sample (mainly readily degradable carbohydrates and glucan and xylan having reduced molecular weights) due to the washing occurs. Therefore, after calculation of the glucan saccharification rate and xylan saccharification rate, a saccharified glucan yield and a saccharified xylan yield were further calculated.

More specifically, the yield on a dry-weight basis of the biomass powder after the alkali treatment was calculated by Equation 5.

The two-step sulfuric acid treatment and saccharification reaction were conducted, and the saccharified glucan yield and saccharified xylan yield of the rice straw after the pretreatment were calculated by Equations 6 and 7.

Meanwhile, in the case where the washing step is not required in neutralization of a sample after the alkali treatment, the glucan saccharification rate and xylan saccharification rate calculated by Equations 3 and 4 were designated as the saccharified glucan yield and saccharified xylan yield, respectively.

Yield on a dry-weight basis (%)=100×dry weight of biomass after alkali treatment/dry weight of biomass feedstock [Eq. 5]

Saccharified glucan yield (%)=100×glucan saccharification rate×yield on a dry-weight basis/(100×glucan content in biomass after alkali treatment/glucan content in biomass feedstock [Eq. 6]

Saccharified xylan yield (%)=100×xylan saccharification rate×yield on a dry-weight basis/(100×xylan content in biomass after alkali treatment/xylan content in biomass feedstock [Eq. 7]

(2) Calculation of the contents of readily degradable carbohydrates in each biomass feedstock

### A. Calculation of Starch Content Per Dry Weight of Biomass

The starch content per dry weight of the biomass was calculated using a Total starch kit (Megazyme).

More specifically, the biomass powder was weighed in an amount of 10 mg and placed in two 1.5 mL plastic tubes. To one of the tubes was added 0.5 mL of water (0.02% NaN₃), and the mixture was stirred vigorously for 10 minutes. After stirring, the sample was immediately cooled to 4° C. and centrifuged (15,000 g, 3 minutes), and part of the supernatant was sampled. The resultant sample was diluted with water, and then the amount of free glucose was measured using a Glucose C-II Test Wako (Wako Pure Chemical Industries, Ltd.) to calculate a free glucose level per dry weight, which was defined as “G value.”

To the other tube were added 300 μL (30 U) of thermostable α-amylase (50 mM MOPS buffer, 0.02% NaN₃, 5 mM CaCl₂, pH 7.0) enzyme solution, and the mixture was treated in a heat block (CTU-N, Taitec) at 100° C. for 10 minutes (vigorously stirred every 2 minutes). After that, the sample was cooled to 50° C., and 400 μL of a sodium acetate buffer (200 mM, 0.02% NaN₃, pH 4.5) and 10 μL (2 U) of an amyloglucosidase enzyme solution were added to conduct a saccharification reaction for 30 minutes while rotating the tube using a thermoblock rotator (SN-48BN, Nissin Scientific Corporation) at 50° C. After the reaction, the sample was immediately cooled to 4° C. and centrifuged (15,000 g, 3 minutes), and part of the supernatant was sampled. The sample was diluted with water, and the amount of glucose was measured using a Glucose C-II Test Wako (Wako Pure Chemical Industries, Ltd.) to calculate a glucose level after the enzymatic reaction per dry weight, which was defined as “StaG value.”

The starch content per dry weight was calculated by subtracting the G value from the StaG value and converting the resulting value into the amount of starch.

B. Calculation of β-(1→3), (1→0.4)-Glucan Content Per Dry Weight of Biomass

The β-(1→3), (1→4)-glucan content per dry weight of the biomass was calculated using a Mixed-linkage Beta-glucan kit (Megazyme).

More specifically, the biomass powder was weighed in an amount of 10 mg and placed in two 1.5 mL plastic tubes. To one of the tubes was added 0.5 mL of water (0.02% NaN₃), and the mixture was stirred vigorously for 10 minutes. After stirring, the sample was immediately cooled to 4° C. and centrifuged (15,000 g, 3 minutes), and part of the supernatant was sampled. The resultant sample was diluted with water, and then the amount of free glucose was measured using a Glucose C-II Test Wako (Wako Pure Chemical Industries, Ltd.) to calculate a free glucose level per dry weight, which was defined as “G value.”

To the other tube were added 480 μL of a sodium acetate buffer (20 mM, pH 5.0), and the mixture was treated in a heat block at 100° C. for 10 minutes (vigorously stirred every 2 minutes). After that, the sample was cooled to 40° C., and 20 μL (1 U) of lichenase were added to conduct a saccharification reaction for 60 minutes while rotating the tube using a thermoblock rotator (SN-48BN, Nissin Scientific Corporation) at 40° C. After the reaction, the sample was immediately cooled to 4° C. and centrifuged (15,000 g, 3 minutes), and 100 μL of the supernatant were sampled. To the sample were added 100 μL of beta-glucosidase (0.23 U, 20 mM, pH 7.0 phosphate buffer) enzyme solution to conduct a saccharification reaction for 30 minutes while rotating the tube using a thermoblock rotator at 40° C. After the reaction, the sample was immediately cooled to 4° C. and centrifuged (15,000 g, 3 minutes), and part of the supernatant was sampled. The sample was diluted with water, and the amount of glucose was measured using a Glucose C-II Test Wako (Wako Pure Chemical Industries, Ltd.) to calculate a glucose level after the enzymatic reaction per dry weight, which was defined as “BetaG value.”

The β-(1→3), (1→4)-glucan content per dry weight was calculated by subtracting the G value from the BetaG value and converting the resulting value into the amount of β-(1→3), (1→4)-glucan.

C. Calculation of Sucrose Content Per Dry Weight of Rice Straw

The sucrose content per dry weight of the rice straw was calculated using a Sucrose, D-fructose and D-glucose kit (Megazyme).

More specifically, rice straw was weighed in an amount of 20 mg and placed in a 1.5 mL plastic tube. To the tube was added 1 mL of water (0.02% NaN₃), and the mixture was stirred vigorously for 10 minutes. After stirring, the sample was immediately cooled to 4° C. and centrifuged (15,000 g, 3 minutes), and 10 μL of the supernatant were sampled. The resultant sample was placed in two wells in a 96-well plate. For the sample in one of the two wells, the amount of free glucose was measured using a Glucose C-II Test Wako (Wako Pure Chemical Industries, Ltd.) to calculate a free glucose level per dry weight, which was defined as “G value.”

To the other well were added 20 μL (4U) of an invertase enzyme solution (citrate buffer, pH 4.6). After the enzyme reaction at 30° C. for 10 minutes, part of the mixture was sampled and diluted with water, and then the amount of free glucose was measured using a Glucose C-II Test Wako (Wako Pure Chemical Industries, Ltd.) to calculate a free glucose level per dry weight, which was defined as “SucG value.”

The sucrose content per dry weight was calculated by subtracting the G value from the SucG value and converting the resulting value into the amount of sucrose.

D. Calculation of Fructose Content Per Dry Weight of Rice Straw

The fructose content per dry weight of rice straw was calculated using a Sucrose, D-fructose and D-glucose kit (Megazyme).

That is, the rice straw was weighed in an amount of 20 mg and placed in a 1.5 mL plastic tube. To the tube was added 1 mL of water (0.02% NaN₃), and the mixture was stirred vigorously for 10 minutes. After stirring, the sample was immediately cooled to 4° C. and centrifuged (15,000 g, 3 minutes), 10 μL of the supernatant was sampled and placed in a 96-plate well. To the well were added 200 μL of water, 10 μL of an imidazole buffer (2M, pH 7.6), and 10 μL of an NADP⁺/ATP (12.5 mg/mL/36.7 mg/mL) aqueous solution, and the mixture was subjected to a reaction at 30° C. for 3 minutes.

After that, an absorbance at 340 nm was measured and defined as an “A1 value.” After measuring the A1 value, 10 μL of a mixed enzyme solution of hexokinase (0.85 U) and glucose-6-phosphate dehydrogenase (0.42 U) were added and the mixture was subjected to a reaction at 30° C. for 10 minutes. Then, an absorbance at 340 nm was measured and defied as an “A2 value” (the absorbance was measured at 2-minute intervals to confirm the stability of the absorbance, and then the subsequent reaction was performed). After measuring the A2 value, 10 μL (2U) of phosphoglucose isomerase were added and the mixture was subjected to a reaction at 30° C. for 10 minutes. Then, an absorbance at 340 nm was measured and defined as an “A3 value.”

A fructose calibration curve of a value obtained by subtracting the A2 value from the A3 value against various concentrations was prepared, and a fructose content per dry weight was calculated.

### Test Example 1

### Optimum pH Range for Saccharification with Enzyme Preparation

First, rice straw powder (variety name: Koshihikari) to be used in the saccharification reaction was subjected to an ammonia treatment (alkali treatment).

More specifically, rice straw powder (50 g) was added to an aqueous 5% (v/v) ammonia solution (1 L), and the mixture was left to stand still at 25° C. for 7 days to conduct the reaction, washed with ultrapure water, and centrifuged (10,000 g, 10 minutes). The procedure was repeated until the pH of the supernatant reached 7.

After a pretreatment subsequent to neutralization with ultrapure water, the rice straw was dried at 60° C. for 3 days. The saccharification reaction was conducted by adding the rice straw powder (50 mg) after the ammonia treatment and 50 mM buffers (1 mL, 0.02% NaN₃) with different pH values to 1.5 mL plastic tubes.

Further, in order to determine an appropriate pH range for saccharification reaction with the enzyme preparation, a glycine buffer (pH 2.0, 2.5, 3.0, 3.5, and 4.0), an acetate buffer (pH 4.0, 4.5, 5.0, 5.5, and 6.0), and a phosphate buffer (pH 6.0, 6.5, 7.0, 7.5, and 8.0) were used as buffers under the respective pH conditions.

As the enzyme preparation, to each of the buffers, a cellulase preparation (12 μL, Celluclast 1.5 L, manufactured by NOVOZYMES JAPAN LTD.), a hemicellulase preparation (6 μL, Ultraflo L, NOVOZYMES JAPAN LTD.), and a β-glucosidase preparation (4 μL, Novozyme 188, Sigma) were added.

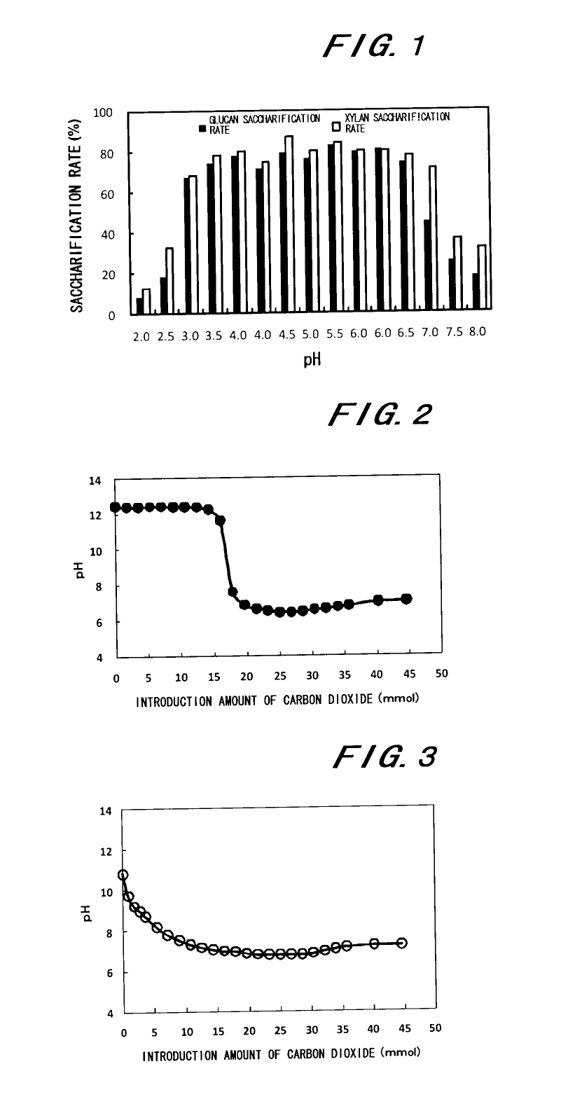

Regarding the reaction conditions, the saccharification reaction was conducted for 24 hours while the tubes were rotated in a thermoblock rotator (SN-48BN, Nissin Scientific Corporation) at 50° C. After the reaction, parts of the mixtures were sampled and diluted with water, and amounts of glucose and amounts of xylose were measured, followed by calculation of glucan saccharification rates and xylan saccharification rates according to the method described in Measurement Example 1 above. FIG. 1 shows the results.

As a result, the total glucan content and total xylan content in the rice straw after the ammonia treatment were found to be 39.8% and 17.6%, respectively (the total glucan content and total xylan content in an untreated rice straw starting material were found to be 31.5% and 14.5%, respectively).

In addition, FIG. 1 shows the glucan saccharification rates and xylan saccharification rates. In general, hydrolase has an optimum pH shifted to the acidic side. However, under enzyme reaction conditions concerning the enzyme preparations used in this experiment and the amounts of the enzyme preparations, the optimum pH range for saccharification of glucan was found to range from 3.0 to 6.5. The saccharification rates rapidly decreased when the pH was less than 3.0 or more than 6.5. On the other hand, the optimum pH range for saccharification of xylan was found to range from 3.0 to 7.0, and the activity at about a neutral pH of 7 was maintained compared with the optimum pH for saccharification of glucan.

The results reveal that, when an appropriate enzyme preparation is used, the neutralization reaction of the biomass after the alkali treatment has only to be conducted until the pH reaches 7.0 or less in the case where the main purpose is saccharification of xylan, or until the pH reaches about pH 6.5 in the case where the main purpose is saccharification of glucan.

### Test Example 2

### Neutralization of Calcium Hydroxide (Ca(OH)₂) Suspension Solution with Carbon Dioxide

Calcium hydroxide is neutralized with carbon dioxide, as in the following reaction equation, to precipitate as calcium carbonate.

Ca(OH)₂+CO₂→CaCO₃↓+H₂O [Chem. 1]

Then, in order to determine the neutralization efficiency of calcium hydroxide by carbon dioxide, a carbon dioxide gas was introduced at a flow rate of 20 mL per minute (0.9 mmol/min) while 100 mL of a calcium hydroxide suspension (1% (w/v), 13.5 mmol) was stirred (100 rpm), and a pH variation was measured with time using a pH meter. Further, at the time of 30 minutes when neutralization with carbon dioxide was completed and the pH became stable at pH 6.3, the introduction of carbon dioxide was stopped, and a pH variation was monitored only with stirring. FIG. 2 shows the results.

As a result, the suspension was neutralized to pH 7 by introducing 18 mmol of carbon dioxide, and the neutralization was achieved by an introduction amount near 13.5 mmol as the theoretical value. In addition, when carbon dioxide was introduced in an amount of 27 mmol, the pH was able to decrease to 6.4. When introduction of carbon dioxide was stopped, an increase in pH (to pH 7) was observed.

### Example 1

### Neutralization with Carbon Dioxide in Open System after Treatment of Rice Straw with Calcium Hydroxide

The efficiency of neutralization with carbon dioxide in an open system was determined using a rice straw suspension subjected to an alkali treatment with calcium hydroxide.

First, 100 mL of a calcium hydroxide suspension (1% (w/v), 13.5 mmol, corresponding to 10% per dry weight of rice straw) and rice straw powder (variety name: Koshihikari, 10 g) were added to a 200 mL glass beaker, and the slurry was homogenized by stirring at room temperature. Then, a treatment with calcium hydroxide (alkali treatment) was conducted at 120° C. for 1 hour using a high-temperature and high-pressure sterilizer (KS-323, Tomy), and the slurry was cooled at room temperature.

After that, a carbon dioxide gas was introduced into the slurry at a flow rate of 20 mL per minute (0.9 mmol/min), and a pH variation was measured with time using a pH meter. Further, at the time of 32 minutes when neutralization with carbon dioxide was completed and the pH became stable at pH 6.76, the introduction of carbon dioxide was stopped, and a pH variation was monitored only with stirring. FIG. 3 shows the results.

As shown in the figure, the rice straw suspension after the treatment with calcium hydroxide was neutralized to pH 7 by introducing 14.3 mmol of carbon dioxide. This suggests that the amount of carbon dioxide necessary for neutralization was small compared with the case where only the calcium hydroxide suspension was neutralized without adding a biomass feedstock (Test Example 2: the amount of carbon dioxide added until the pH reached 7 was 18 mmol).

This phenomenon is probably caused by the fact that alkali metal cations (such as Na⁺, Ca²⁺, and Mg²⁺) are bonded to acidic groups (mainly a carboxyl group (—COOH) in hemicellulose and a phenol group in lignin) in the rice straw, resulting in a decrease in the amounts of the cations present in the aqueous solution.

In addition, it was found that, when carbon dioxide was introduced in an amount of 23.1 mmol, the pH was able to decrease to a constant value of pH 6.76, and when introduction of carbon dioxide was stopped, the pH increased (to pH 7.22).

### Example 2

### Neutralization with Carbon Dioxide in Closed System after Treatment of Rice Straw with Calcium Hydroxide

The ability of neutralization with carbon dioxide in a closed system was determined using a rice straw suspension subjected to an alkali treatment with calcium hydroxide.

First, 4 mL of each of calcium hydroxide suspensions with different concentrations (0, 0.1, 0.5, 1.0, 2.0, and 4.0% (w/v) corresponding to 0, 2, 10, 20, 40, and 80% (w/w) per dry weight of rice straw) and rice straw powder (variety name: Koshihikari, 200 mg) were added to a 10 mL vial bottle (No. 3, Maruemu Corporation). The vial bottle was sealed with a butyl rubber stopper and an aluminum cap, and the slurry was homogenized by stirring. Then, a treatment with calcium hydroxide (alkali treatment) was conducted at 120° C. for 1 hour using a high-temperature and high-pressure sterilizer, and the slurry was cooled at room temperature.

It should be noted that, the pH measurement after each treatment with calcium hydroxide was conducted by sampling 50 μL of calcium hydroxide treatment solution from the vial bottle using a 1 mL syringe (SS-01T, Terumo Corporation) and a needle (NN-2138R, 0.80×38 mm, Terumo Corporation).

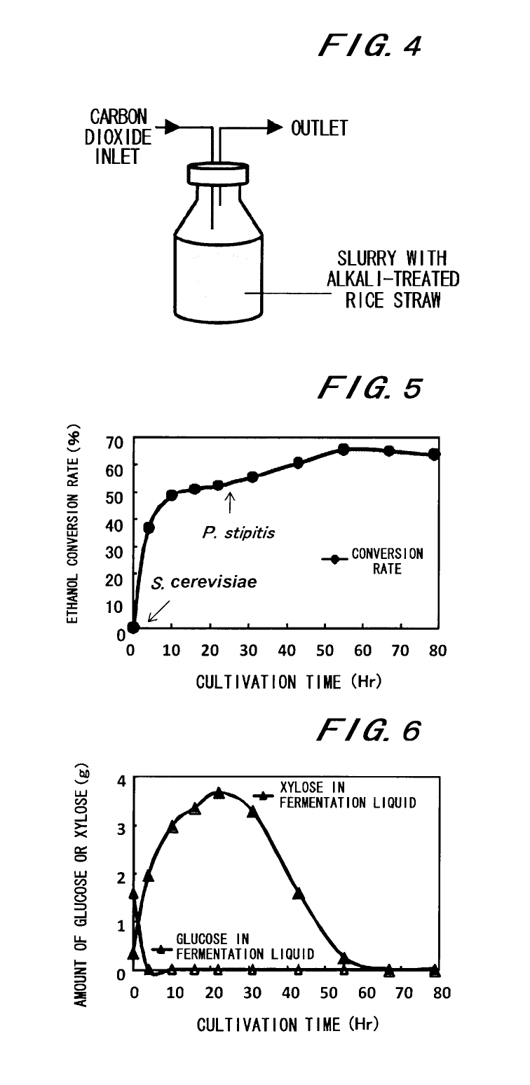

After that, neutralization in the closed system was conducted by a method involving: replacing the gas phase in the vial bottle by a carbon dioxide gas (500 mL/min, 0.15 MPa), which had been passed through a sterile filter (0.45 μm), for 20 seconds using two needles (NN-2138R, NN-2070C, Terumo Corporation) as illustrated in FIG. 4; removing the needle on the outlet side (NN-2138R); placing the needle on the inlet side (NN-2070C) in the liquid; and pressurizing the solution at a carbon dioxide pressure of 0.15 MPa in the vial bottle for 20 minutes.

50 μL of the neutralization reaction solution in the vial bottle were sampled using a 1 mL syringe (SS-01T) and a needle (NN-2138R), and the pH in the neutralization reaction solution after neutralization with carbon dioxide was measured immediately using a pH meter. This step was conducted aseptically in a clean bench. Table 1 shows the results.

TABLE 1

| Concentration of | pH after treatment | pH after |
|---|---|---|
| calcium hydroxide | with calcium | neutralization with |
| (% (w/w))¹ | hydroxide | carbon dioxide |
| 0 | 6.1 | 5.1 |
| 2 | 6.8 | 5.4 |
| 10 | 8.9 | 6.3 |
| 20 | 10.3 | 6.4 |
| 40 | 12.2 | 6.2 |
| 80 | 12.2 | 6.5 |

Alkali treatment conditions: calcium hydroxide, 120° C., 1 hour
¹% (w/w) = 100 × calcium hydroxide g/biomass g

As shown in the table, the pH after the treatment with calcium hydroxide (alkali treatment) increased with increasing the concentration of calcium hydroxide, but the pH after neutralization with carbon dioxide was pH 6.5 or less in all the cases. It should be noted that the values were lower compared with the case of neutralization in the open system (Example 1: pH after neutralization: 6.76). This is probably caused by an increase in the concentration of a carbonate ion in the reaction solution by a partial pressure of carbon dioxide in the gas phase.

In consideration of the optimum pH range of the enzyme preparation in Test Example 1, it was found that, when neutralization with carbon dioxide was conducted after the treatment with calcium hydroxide in the closed system, the pH was easily adjusted to a value suitable for the glucan saccharification reaction and xylan saccharification reaction.

### Example 3

### Neutralization with Carbon Dioxide in Fermenter after Treatment of Rice Straw with Calcium Hydroxide

For a rice straw suspension subjected to an alkali treatment with calcium hydroxide in a fermenter, the ability of neutralization with carbon dioxide was examined.

First, 450 mL of a calcium hydroxide suspension (4%, corresponding to 36% per dry weight of the rice straw) and rice straw powder (50 g) were added to a 1 L glass bottle, and the slurry was homogenized by stirring. The slurry was subjected to a treatment with calcium hydroxide (alkali treatment) using a high-temperature and high-pressure sterilizer at 120° C. for 1 hour, and cooled at room temperature.

After that, the rice straw suspension after the treatment with calcium hydroxide was placed in a 1 L fermenter (type Bioneer-C, B. E. MARUBISHI Co., Ltd., previously sterilized at a high temperature and high pressure at 121° C. for 10 minutes). In this procedure, the 1 L glass bottle was washed with 50 mL of sterile water twice, and all the washing solutions were placed in the 1 L fermenter. This step was conducted aseptically in a clean bench. After that, a pH variation in the fermenter was monitored while stirring the suspension (400 rpm) and introducing carbon dioxide (100 mL/min).

As a result, 40 minutes after introduction of carbon dioxide, the pH in the fermenter decreased to 6.1, and then was maintained stably at pH 6.1.

In consideration of the optimum pH range of the enzyme preparation in Test Example 1, it was found that, similar to the example of neutralization in the closed system of Example 2, the pH was easily adjusted to a value suitable for the glucan saccharification reaction and xylan saccharification reaction even in the fermenter.

### Test Example 3

### Enzymatic Saccharification after Treatment with Calcium Hydroxide, Neutralization with Hydrochloric Acid, and Washing with Water of Rice Straw

(1) Treatment with Calcium Hydroxide, Neutralization with Hydrochloric Acid, and Washing with Water

Rice straw subjected to the treatment with calcium hydroxide was neutralized with hydrochloric acid, and washed with water.

First, 10 mL of each of calcium hydroxide suspensions with different concentrations (0, 0.1, 0.5, 1.0, 2.0, and 4.0% (w/v) corresponding to 0, 2, 10, 20, 40, and 80% (w/w) per dry weight of rice straw) and rice straw powder (variety name: Koshihikari, 500 mg) were added to a 30 mL glass bottle, and the slurry was homogenized by stirring. Then, a treatment with calcium hydroxide (alkali treatment) was conducted at 120° C. for 1 hour using a high-temperature and high-pressure sterilizer, and the slurry was cooled at room temperature.

After that, the slurry was neutralized with hydrochloric acid (1 M), and the pH was lowered to 1, to thereby convert an excessive amount of calcium hydroxide into calcium chloride. Subsequently, the slurry was transferred to a 15 mL plastic tube, washed with ultrapure water, and centrifuged (16,000 g, 10 minutes), and the steps were repeated until the pH of the supernatant became 4.5 or more.

Then, the resultant solid matter (pellet) collected after neutralization and washing with water was dried at 75° C. for 1 day, and the dry weight of the pellet was measured.

### (2) Saccharification Reaction

50 mg of the solid matter obtained through the above-mentioned steps (rice straw pellet neutralized with hydrochloric acid and washed after treatment with calcium hydroxide) were weighed in a 1.5 mL plastic tube, and a 50 mM citrate buffer (1 mL, pH 4.8, 0.02% NaN₃) were added together with a cellulase preparation (12 μL, Celluclast 1.5 L, NOVOZYMES JAPAN LTD.), a hemicellulase preparation (6 μL, Ultraflo L, NOVOZYMES JAPAN LTD.), and a (3-glucosidase preparation (20 μL, Novozyme 188, Sigma) as enzyme preparations.

Regarding enzymatic reaction conditions, the saccharification reaction was conducted for 24 hours while the tubes were rotated in a thermoblock rotator (SN-48BN, Nissin Scientific Corporation) at 50° C. After the reaction, parts of the mixtures were sampled and diluted with water, and amounts of glucose and amounts of xylose were measured according to the method described in Measurement Example 1.

Further, a two-step sulfuric acid treatment was conducted for the rice straw starting material and rice straw after the treatment with calcium hydroxide, and saccharified glucan yields and saccharified xylan yields were calculated according to the method described in Measurement Example 1.

Table 2 shows the results.

TABLE 2

| | Yield | | | | |
|---|---|---|---|---|---|
| Concentration | on | | | | |
| of | a dry- | | | | |
| calcium | weight | Glucan | Saccharified | Xylan | Saccharified |
| hydroxide | basis | content | glucan yield | content | xylan yield |
| (% (w/w))¹ | (%) | (%) | (%) | (%) | (%) |
| 0 | 85.6 | 35.8 | 29.2 | 15.7 | 14.0 |
| 2 | 86.1 | 37.2 | 35.8 | 14.7 | 20.5 |
| 10 | 80.6 | 38.4 | 59.1 | 14.8 | 44.4 |
| 20 | 75.2 | 41.8 | 73.5 | 15.3 | 49.7 |
| 40 | 74.8 | 41.0 | 71.3 | 15.0 | 48.4 |
| 80 | 73.3 | 42.3 | 77.8 | 15.0 | 49.0 |

Alkali treatment conditions: calcium hydroxide, 120° C., 1 hour
¹% (w/w) = 100 × calcium hydroxide g/biomass g

As shown in the results in the table, the yield on a dry-weight basis decreased from 85.6% to 73.3% with increasing the concentration of calcium hydroxide in the treatment with calcium hydroxide. However, the glucan content tended to increase from 35.8% (31.5% in the case of the untreated rice straw starting material) to 42.3%, and the glucan yield tended to increase from 29.2% to 77.8%.

On the other hand, the xylan contents were relatively constant (about 15%) compared with the glucan contents, and in consideration of a decrease in the yield on a dry-weight basis, xylan having a reduced molecular weight obtained in the treatment with calcium hydroxide was probably lost by the washing step. In addition, although the xylan yields increased (from 14.0% to 49.0%), the yields were lower than the glucan yields.

### Example 4

### Enzymatic Saccharification after Treatment with Calcium Hydroxide and Neutralization with Carbon Dioxide of Rice Straw

For each slurry of the rice straw which had been subjected to the treatment with calcium hydroxide and the step of neutralization with carbon dioxide in a closed system, prepared in Example 2, a saccharification reaction was conducted.

That is, a cellulase preparation (48 μL, Celluclast 1.5 L, NOVOZYMES JAPAN LTD.), a hemicellulase preparation (24 μL, Ultraflo L, NOVOZYMES JAPAN LTD.), and a β-glucosidase preparation (80 μL, Novozyme 188, Sigma) as enzyme preparations were passed together with ultra pure water (848 μL) through a sterile filter (0.45 μm), and then injected into a vial bottle (see Example 2), in which the slurry prepared in Example 2 (i.e., the slurry neutralized with carbon dioxide) was placed, using a 1 mL syringe (SS-01T, Terumo Corporation) and a needle (NN-2138R, 0.80×38 mm, Terumo Corporation). This step was conducted aseptically in a clean bench.

Regarding reaction conditions, the enzymatic saccharification reaction was conducted for 24 hours while the vial bottles were rotated using a rotator (RKVSD, ATR) in an incubator at 50° C.

After the saccharification reaction, part of the mixture was sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1. Table 3 shows the results.

TABLE 3

| Concentration of | Saccharified glucan | Saccharified xylan |
|---|---|---|
| calcium hydroxide | yield | yield |
| (% (w/w))¹ | (%) | (%) |
| 0 | 34.5 | 20.1 |
| 2 | 44.0 | 33.4 |
| 10 | 69.1 | 57.4 |
| 20 | 74.2 | 64.3 |
| 40 | 72.8 | 64.4 |
| 80 | 77.0 | 65.8 |

Alkali treatment conditions: calcium hydroxide, 120° C., 1 hour
¹% (w/w) = 100 × calcium hydroxide g/biomass g

As shown in the table, the saccharified glucan yield tended to increase (from 34.5% to 77.0%) with increasing the concentration of calcium hydroxide. In addition, as compared with the saccharified glucan yields in the hydrochloric acid neutralization method of Test Example 3, the saccharified glucan yields were higher at calcium hydroxide concentrations of 0, 2, 10, 20, 40, and 80%, which revealed that the saccharified glucan yield at the calcium hydroxide concentration of 80% was almost the same as that in the hydrochloric acid neutralization method.

On the other hand, the saccharified xylan yield tended to increase (from 20.1% to 65.8%) with increasing the concentration of calciumhydroxide . In addition, as compared with the hydrochloric acid neutralization method of Test Example 3, the saccharified xylan yields were higher by about 15% than those obtained in the hydrochloric acid neutralization method at all the calcium hydroxide concentrations.

### Example 5

### Enzymatic Saccharification after Alkali Treatment with Different Alkalis and Neutralization with Carbon Dioxide

In the cases where rice straw was subjected to various alkali treatments using different alkali solutions, enzymatic saccharification reactions after neutralization with carbon dioxide were examined.

First, rice straw powder (variety name: Koshihikari, 200 mg) was added to solutions (4 mL) of 270 mM (corresponding to the concentration of calcium hydroxide of 80% per dry weight of the rice straw) alkalis (calcium hydroxide, sodium hydroxide, potassium hydroxide, and magnesium hydroxide). Then, the alkali treatment was conducted in the same manner as in the method described in Example 2 except that the treatment was conducted using such alkali solutions under conditions of 120° C. for 2 hours, and neutralization with carbon dioxide and pH measurement were conducted. Subsequently, enzymatic saccharification reactions were conducted in the same manner as in the method described in Example 4.

After the saccharification reaction, part of the mixture was sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1.

Table 4 shows the pHs after neutralization with carbon dioxide, saccharified glucan yields, and saccharified xylan yields.

TABLE 4

| | pH after | | |
|---|---|---|---|
| | neutralization | | |
| | with carbon | Saccharified | Saccharified |
| Alkali | dioxide | glucan yield (%) | xylan yield (%) |
| Calcium | 6.1 | 75.8 | 68.1 |
| hydroxide | | | |
| Potassium | 7.0 | 61.7 | 61.3 |
| hydroxide | | | |
| Sodium | 7.0 | 51.8 | 47.0 |
| hydroxide | | | |
| Magnesium | 6.4 | 25.1 | 19.4 |
| hydroxide | | | |

Alkali treatment conditions: corresponding to 80% calcium hydroxide (w/w), 120° C., 2 hours

The pH after neutralization with carbon dioxide was found to be pH 7 or less in all the alkali treatment systems, and the value was the lowest (pH 6.1) in the system using calcium hydroxide.

Further, the results of the saccharified glucan yield and saccharified xylan yield suggest that the saccharification reaction after neutralization with carbon dioxide can be conducted in the alkali treatment systems using potassium hydroxide and sodium hydroxide, but the system using calcium hydroxide was found to have the highest yields (75.8%, 68.1%).

It should be noted that, the alkali treatment (treatment with calcium hydroxide) was conducted for 2 hours in this example, but a significant effect of increasing the yield by a treatment time was not obtained compared with the case where the 80% calcium hydroxide treatment was conducted for 1 hour in Example 4 (saccharified glucan yield: 77.0%, saccharified xylan yield: 65.8%).

### Example 6

### Enzymatic Saccharification after Treatment with Calcium Hydroxide and Neutralization with Carbon Dioxide of Different Biomasses

Enzymatic saccharification reactions were conducted after treatments with calcium hydroxide and neutralization with carbon dioxide of different biomass powders.

First, 4 mL of a1% calcium hydroxide suspension (corresponding to 20% per dry weight of each biomass) and various biomasses [rice straw (variety name: Koshihikari), sugarcane bagasse (available from a sugar factory in Japan), barley straw (variety name: Silky Snow), and sorghum bagasse (variety name: SIL-05)] powders (200 mg) were added, and the mixtures were subjected to a treatment with calcium hydroxide (alkali treatment) using a metallic portable reactor (type TYS-1, Taiatsu Techno) in an oil bath at 160° C. for 2 hours, and cooled at room temperature.

After that, the wholes of the treated products were placed in 10 mL vial bottles, and neutralized with carbon dioxide in the same manner as in the method described in Example 2, and enzymatic saccharification reactions were conducted in the same manner as in the method described in Example 4.

After the saccharification reaction, parts of the mixtures were sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1. Table 5 shows the results.

TABLE 5

| | Glucan | Xylan | Saccharified | Saccharified |
|---|---|---|---|---|
| Biomass | content | content | glucan yield | xylan yield |
| feedstock | (%) | (%) | (%) | (%) |
| Rice straw | 31.5 | 14.5 | 73.5 | 83.2 |
| Sugarcane | 36.8 | 21.9 | 71.7 | 85.2 |
| bagasse | | | | |
| Barley straw | 28.2 | 13.4 | 67.1 | 82.8 |
| Sorghum | 32.4 | 17.7 | 72.9 | 87.7 |
| bagasse | | | | |

Alkali treatment conditions: 20% calcium hydroxide (w/w), 160° C., 2 hours

As shown in the table, the glucan content and xylan content varied depending on the type of the biomass feedstock, and the sugarcane bagasse was found to show the highest contents (36.8%, 21.9%). In all the biomasses, the saccharified glucan yields were found to be about 70%.

It should be noted that, in this example, the treatment with calcium hydroxide was conducted at 160° C. for 2 hours, but as compared with the case where the rice straw was treated with 1% calcium hydroxide (corresponding to 20% per dry weight of the rice straw) at 120° C. for 1 hour in Example 4 (saccharified glucan yield: 74.2%, saccharified xylan yield: 64.3%), the saccharified xylan yield was found to be 83.2% and the yield increased by about 20%, although a significant effect of increasing the saccharified glucan yield by an increase in temperature was not obtained.

In addition, in view of the fact that the treatment time (a difference between 1 hour and 2 hours) has little effect on the yield as shown in Example 5, the ‘treatment temperature’ is considered to be an important factor in a step which requires a high xylan yield.

### Example 7

### Enzymatic Saccharification after Treatment with Calcium Hydroxide and Neutralization with Carbon Dioxide of Rice Straw Containing Readily Degradable Carbohydrates

### (1) Contents of Readily Degradable Carbohydrates, Glucan Content, and Xylan Content in Rice Straw

Rice straw contains not only cellulose and hemicellulose but also many readily degradable carbohydrates (glucose, sucrose, fructose, starch, and β-(1→3), (1→4)-glucan). The contents of such readily degradable carbohydrates vary depending on the variety, harvest season, and preservation method of the rice straw. The contents of readily degradable carbohydrates, glucan content, and xylan content in the rice straw were measured according to the methods described in Measurement Examples 1 and 2. Table 6 shows the results.

TABLE 6

| Rice straw | Glucose | Fructose | Sucrose | Starch | β-(1→3),(1→4)- | Glucan | Xylan |
|---|---|---|---|---|---|---|---|
| (variety | content | content | content | content | glucan content | content | content |
| name) | (%) | (%) | (%) | (%) | (%) | (%) | (%) |
| Koshihikari | 0.0 | 0.0 | 0.0 | 2.2 | 0.3 | 31.5 | 14.5 |
| Leaf Star | 0.8 | 0.9 | 3.5 | 20.8 | 1.5 | 46.2 | 9.2 |

The table shows that the variety Leaf Star has a particularly high starch content and contains sucrose at a high content.

(2) Enzymatic Saccharification after Treatment with Calcium Hydroxide and Neutralization with Carbon Dioxide of Rice Straw Containing Readily Degradable Carbohydrates

The readily degradable carbohydrates are lost in a washing step in a conventional alkali treatment. However, the washing step is not used in saccharification after a pretreatment with calcium hydroxide and neutralization with carbon dioxide, and hence the loss does not occur.

Therefore, the enzymatic saccharification reaction was conducted after the pretreatment with calcium hydroxide and neutralization with carbon dioxide using rice straw containing such readily degradable carbohydrates. Meanwhile, as comparative data, enzymatic saccharification was conducted after the treatment with calcium hydroxide, neutralization with hydrochloric acid, and washing with water.

First, two vial bottles in which rice straw (200 mg) was added to 4 mL of a 1% calcium hydroxide suspension (corresponding to 20% per dry weight of the rice straw) were prepared.

In one bottle, the treatment with calcium hydroxide (120° C., 1 hour) and neutralization with carbon dioxide were conducted in the same manner as in the method described in Example 2, and the enzymatic saccharification reaction was conducted in the same manner as in the method described in Example 4.

In the other bottle, as a comparative control, the treatment with calcium hydroxide (120° C., 1 hour) was conducted according to Example 2, and the enzymatic saccharification reaction was conducted after neutralization with hydrochloric acid and washing with water in the same manner as in the method described in Test Example 3.

After the saccharification reaction, parts of the mixtures were sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1.

Table 7 shows the results.

TABLE 7

| | | | Saccharification after | |
|---|---|---|---|---|
| | Saccharification after | | neutralization with | |
| | neutralization with | | hydrochloric acid and | |
| | carbon dioxide | | washing with water | |
| Rice straw | Saccharified | Saccharified | Saccharified | Saccharified |
| (variety | glucan yield | xylan yield | glucan yield | xylan yield |
| name) | (%) | (%) | (%) | (%) |
| Koshihikari | 74.2 | 64.3 | 73.5 | 49.7 |
| Leaf star | 83.6 | 61.7 | 58.2 | 51.3 |

Alkali treatment conditions: 20% calcium hydroxide (w/w), 120° C., 1 hour

The table shows that, in the case of Leaf Star containing readily degradable carbohydrates at a high content, the saccharified glucan yield and saccharified xylan yield of the saccharification reaction after neutralization with carbon dioxide were higher than those of the saccharification reaction after neutralization with hydrochloric acid and washing with water.

The results reveal that saccharification after neutralization with carbon dioxide is suitable as a pretreatment step of the saccharification reaction of rice straw containing readily degradable carbohydrates.

It should be noted that, although a starch degrading enzyme was not added as an enzyme preparation, the starch in Leaf Star was degraded probably because the enzyme preparation added as a β-glucosidase preparation (Novozyme 188, Sigma) has a high activity to degrade starch.

(3) Measurement of Amounts of Sucrose and Starch Lost in Neutralization with Hydrochloric Acid and Washing with Water after Calcium Hydroxide Pretreatment

Further, amounts of sucrose and starch lost in neutralization with hydrochloric acid and washing with water after calcium hydroxide pretreatment were measured.

First, after the treatment with calcium hydroxide, the sample was neutralized with hydrochloric acid and centrifuged (16,000 g, 10 minutes) to collect the supernatant, and amounts of sucrose and starch in 4 mL of the supernatant were measured to calculate the contents (%) per dry weight of the untreated rice straw starting material. Table 8 shows the results. It should be noted that, the contents of the readily degradable carbohydrates lost are values per dry weight of the rice straw before the alkali treatment.

TABLE 8

| Rice straw | | |
|---|---|---|
| (variety name) | Sucrose content (%) | Starch content (%) |
| Koshihikari | 0.0 | 0.7 |
| Leaf star | 3.3 | 4.8 |

The results reveal that the supernatant obtained by the calcium hydroxide pretreatment of Leaf Star contained 3.3% sucrose and 4.8% starch. The sucrose amount is comparable to the total sucrose content in Leaf Star before the treatment with calcium hydroxide. That is, the results show that sucrose was completely washed away by repeating the washing step.

Meanwhile, about 20% of the whole starch was washed away, and it was predicted that a larger amount of starch was washed away by repeating the washing step in view of the property of starch gelatinized by the heat treatment.

Those facts show that the method involving conducting saccharification after neutralization with carbon dioxide without washing before the treatment with calcium hydroxide is suitable as a pretreatment method conducted after the saccharification reaction.

It should be noted that, the supernatant of Leaf Star was found to contain 3.3% sucrose even after the harsh treatment with calcium hydroxide at 120° C. for 1 hour.

### Example 8

### Enzymatic Saccharification after Treatment with Calcium Hydroxide and Neutralization with Carbon Dioxide of Sugarcane

Two vial bottles were prepared, in each of which sugarcane powder (variety name: Nif8, 200 mg) obtained by drying harvested sugarcane at 60° C., followed by pulverization was added to 4 mL of a 1% calcium hydroxide suspension (corresponding to 20% per dry weight of the sugarcane).

In one bottle, the treatment with calcium hydroxide (120° C., 1 hour) and neutralization with carbon dioxide were conducted in the same manner as in the method described in Example 2, and the enzymatic saccharification reaction was conducted in the same manner as in the method described in Example 4.

In the other bottle, as a comparative control, the treatment with calcium hydroxide (120° C., 1 hour) was conducted in the same manner as in the method described in Example 2, and then the neutralization with hydrochloric acid, washing with water, and enzymatic saccharification reaction was conducted in the same manner as in the method described in Test Example 3.

After the saccharification reaction, parts of the mixtures were sampled and diluted with water, and amounts of glucose, amounts of xylose, and fructose were measured according to the method described in Measurement Example 1.

Meanwhile, regarding sucrose contents, an ‘untreated sugarcane starting material’ and ‘sugarcane washed with water to remove sucrose and dried without conducting the treatment with calcium hydroxide’ were subjected to the two-step sulfuric acid treatment, and the sucrose contents were measured according to the method described in Measurement Example 1.

Further, the saccharified glucan yield and saccharified xylan yield were calculated according to the method described in Measurement Example 1. However, the amount of glucose in Equation 1 was calculated by adding the sucrose content converted into glucose to the amount of glucose obtained by the two-step sulfuric acid treatment. The amount of enzymatically saccharified glucose in Equation 3 was calculated by adding fructose generated by the enzymatic saccharification reaction converted into the same amount of glucose.

Table 9 shows the results.

TABLE 9

| | | | Saccharification after | |
|---|---|---|---|---|
| | Saccharification after | | neutralization with | |
| | neutralization with | | hydrochloric acid and | |
| | carbon dioxide | | washing with water | |
| | Saccharified | Saccharified | Saccharified | Saccharified |
| Sugarcane | glucan yield | xylan yield | glucan yield | xylan yield |
| (variety name) | (%) | (%) | (%) | (%) |
| Nif8 | 84.8 | 51.7 | 27.5 | 56.5 |

Alkali treatment conditions: 20% calcium hydroxide (w/w), 120° C., 1 hour

The table shows that the saccharified glucan yield in saccharification after the treatment with calcium hydroxide and neutralization with carbon dioxide of sugarcane containing sucrose (15.6% per dry weight) was 84.8%. The value was three times or more as high as that of the method of saccharification after neutralization with hydrochloric acid and washing with water.

These results correspond to those of Example 7, which show that sucrose included in the rice straw was not degraded by the treatment with calcium hydroxide, and suggest that it is effective to saccharify a biomass containing sucrose at a high content, such as sugarcane, after the treatment with calcium hydroxide and neutralization with carbon dioxide.

### Example 9

### Enzymatic Saccharification after Preservation in Calcium Hydroxide and Neutralization with Carbon Dioxide of Rice Straw

Calcium hydroxide and water were added to rice straw, and the mixture was preserved at 30° C. and appropriately further subjected to a heat treatment to examine the enzymatic saccharification ability of the rice straw suspension preserved after neutralization with carbon dioxide.

More specifically, 200 mg of rice straw, 40 mg of calcium hydroxide, and 4 mL of water were added to 10 mL vial bottles, and the bottles were sealed and stirred according to Example 2, to thereby prepare slurries. The slurries were left to stand still and preserved at 30° C. for 3 days or 6 days before the heat treatment. After that, the vial bottles containing the slurries preserved for 3 days or 6 days were subjected to heat treatments at 30° C., 60° C., 90° C., 120° C., and 150° C., respectively, for 1 hour and cooled at room temperature to conduct the treatment with calcium hydroxide, and neutralization with carbon dioxide and pH measurement were conducted in the same manner as in the method described in Example 2. Then, the enzymatic saccharification reaction was conducted in the same manner as in the method described in Example 4.

After the saccharification reaction, parts of the slurries were sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1. Table 10 shows the results.

TABLE 10

| Temperature | Three-day preservation | | Six-day preservation | |
|---|---|---|---|---|
| (° C.) | treatment | | treatment | |
| in heat | Saccharified | Saccharified | Saccharified | Saccharified |
| treatment | glucan yield | xylan yield | glucan yield | xylan yield |
| (1 hour) | (%) | (%) | (%) | (%) |
| 30 | 66.4 | 66.2 | 76.0 | 65.2 |
| 60 | 72.2 | 64.4 | 75.9 | 63.3 |
| 90 | 75.0 | 66.4 | 75.0 | 64.1 |
| 120 | 71.7 | 64.0 | 71.1 | 63.0 |
| 150 | 64.0 | 62.6 | 70.8 | 63.5 |

Alkali treatment conditions: corresponding to 20% calcium hydroxide (calcium hydroxide w/rice straw w)

The table shows that the yield in the case of long-term preservation in calcium hydroxide was almost the same as that in the case of the treatment with calcium hydroxide by the high-temperature and high pressure treatment in Example 4.

Further, the table shows that, after long-term preservation, a significant difference of the yields was not caused by the presence or absence of the heat treatment.

### Example 10

### Effect of Grinding of Slurry Before and after Neutralization with Carbon Dioxide on Saccharification Efficiency

Slurries before and after neutralization with carbon dioxide were ground, and the effect of grinding on saccharification was examined.

First, rice straw powder (variety name: Koshihikari, 4 g), calcium hydroxide (800 mg), and water (40 mL) were added to three 50 mL vial bottles (Maruemu Corporation), and the vial bottles were sealed with butyl rubber steppers and aluminum caps, followed by stirring to homogenize slurries (corresponding to 20% calcium hydroxide (w/w, calcium hydroxide g/rice straw g)). One of the bottles was subjected to a treatment with calcium hydroxide at 120° C. for 1 hour using a high-temperature and high-pressure sterilizer and cooled at room temperature so as to serve as a sample treated with calcium hydroxide. The other two bottles were subjected to a treatment with calcium hydroxide by static preservation in calcium hydroxide at 30° C. for 3 days or 6 days.

After that, the ‘slurry after the high-temperature and high pressure treatment (120° C., 1 hour)’ and the ‘slurry after the three-day calcium hydroxide preservation treatment’ were subjected to a five-time grinding treatment using a grinder mill (MICRO POWDER, WEST), and suspensions prepared so as to contain 200 mg of the rice straw powder and 4 mL of water were added to 10 mL vial bottles. After that, the vial bottles were subjected to neutralization with carbon dioxide and pH measurement in the same manner as in the method described in Example 2. Then, the enzymatic saccharification reaction was conducted in the same manner as in the method described in Example 4.

Meanwhile, the ‘slurry after the six-day calcium hydroxide preservation treatment’ was subjected to neutralization with carbon dioxide and pH measurement in the same manner as in the method described in Example 2, and then ground using the grinder mill five times. Then, suspensions prepared so as to contain 200 mg of the rice straw powder and 4 mL of water were added to 10 mL vial bottles. Further, the enzymatic saccharification reaction was conducted in the same manner as in the method described in Example 4 except that hygromycin B (H772-1G, Sigma, 2.5 mg) serving as an antibiotic was added.

After the saccharification reaction, parts of the suspensions were sampled and diluted with water, and amounts of glucose and amounts of xylose were measured according to the method described in Measurement Example 1. In addition, the two-step sulfuric acid treatment was conducted for the untreated rice straw starting material. Then, saccharified glucan yields and saccharified xylan yields were calculated according to the method described in Measurement Example 1. Table 11 shows the results.

TABLE 11

| | Steps after | Saccharified | Saccharified |
|---|---|---|---|
| Treatment with | treatment with | glucan yield | xylan yield |
| calcium hydroxide | calcium hydroxide | (%) | (%) |
| High-temperature | Grinding → | 83.7 | 70.4 |
| and high-pressure | Neutralization → | | |
| treatment | Saccharification | | |
| Three-day | Grinding → | 85.3 | 73.1 |
| preservation | Neutralization → | | |
| treatment | Saccharification | | |
| Six-day | Neutralization → | 86.3 | 72.6 |
| preservation | Grinding → | | |
| treatment | Saccharification | | |

Corresponding to 20% calcium hydroxide (w/w, calcium hydroxide g/rice straw g),

The table shows that the saccharified glucan yields and saccharified xylan yields of all the ground samples were improved compared with the samples not ground under reaction conditions of the same concentrations of calcium hydroxide (Example 4). Specifically, the results reveal that the saccharified glucan yield was improved by 10% at a maximum, while the saccharified xylan yield was improved by 8% at a maximum.

### Example 11

### Simultaneous Saccharification and Fermentation after Treatment with Calcium Hydroxide and Neutralization with Carbon Dioxide of Rice Straw

Rice straw was subjected to the treatment with calcium hydroxide and neutralized with carbon dioxide using a 1 L fermenter, to thereby prepare a slurry, which was used as a substrate to conduct simultaneous saccharification and fermentation (fermentation method to simultaneously conduct enzymatic saccharification and ethanol fermentation).

It should be noted that, in this example, in enzyme systems using a cellulase preparation, a hemicellulase preparation, and a β-glucosidase preparation, Saccharomyces cerevisiae NBRC0224 targeting glucan and Pichia stipitis NBRC10063 targeting xylan were used as microorganisms for ethanol fermentation to conduct the simultaneous saccharification and fermentation.

First, a 1 L fermenter (type Bioneer-C, B. E. MARUBISHI Co., Ltd.) in which the rice straw powder was subjected to the treatment with calcium hydroxide (4%, 120° C., 1 hour) and neutralization with carbon dioxide in the same manner as in the method described in Example 3 was prepared.

A cellulase preparation (12 mL, Celluclast 1.5 L, manufactured by NOVOZYMES JAPAN LTD.), a hemicellulase preparation (6 mL, Ultraflo L, manufactured by NOVOZYMES JAPAN LTD.), and a β-glucosidase preparation (16 mL, Novozyme 188, manufactured by Sigma) were passed together with ultrapure water (66 mL) through a sterile filter (0.45 μm), and the passed sample was aseptically added to the fermenter.

After that, 50 mL of a suspension of S. cerevisiae [obtained by preculturing the microorganism in YPD medium at 30° C. for 16 hours, centrifuging the mixture (5,000 g, 10 minutes) to collect the cells, washing/centrifuging the cells with sterile physiological saline twice, and adjusting O.D._{600 nm} to 2 at the start of the simultaneous saccharification and fermentation] was inoculated aseptically into the fermenter.

After inoculation, introduction of carbon dioxide was stopped, and the simultaneous saccharification and fermentation (saccharification reaction and fermentation of ethanol derived from glucan) was conducted at 30° C. while rotating at 200 rpm.

Further, after inoculation, part of the culture in the fermenter was aseptically sampled to measure concentrations of glucose, xylose, and ethanol in the fermenter. Ethanol was quantified by passing a sample liquid through a filter (0.45 μm) and subjecting the resultant to HPLC (LC-20AD, SIL-20AC, CTO-20AC, RID-10A, Shimadzu) and an AminexR HPX-87H column (300 mm×7.8 mm, Bio-Rad).

At the time when production of ethanol derived from glucan reached plateau (24 hours after the start of culture), 50 mL of a suspension of P. stipitis [obtained by preculturing the microorganism in YPX medium at 30° C. for 16 hours, centrifuging the mixture (5,000g, 10 minutes) to collect cells, washing/centrifuging the cells with sterile physiological saline twice, and adjusting O.D.₆₀₀ₙₘ to 2 at the start of the simultaneous saccharification and fermentation] was inoculated aseptically into the fermenter.

After inoculation, the simultaneous saccharification and fermentation (saccharification reaction and fermentation of ethanol derived from xylan) was conducted at 30° C. while introducing air into the suspension (5 mL/min) and rotating the fermenter (200 rpm).

Further, after inoculation, part of the culture in the fermenter was sampled aseptically to measure concentrations of glucose, xylose, and ethanol in the fermenter.

A rate of conversion of glucan into ethanol by S. cerevisiae in a period from the start of the simultaneous saccharification and fermentation to 22 hours after the start, a rate of conversion of xylan into ethanol by P. stipitis after 22 hours or later, and a total ethanol conversion rate were calculated by the two-step sulfuric acid treatment method and by the following equations 8, 9, and 10. FIG. 5 shows the results. It should be noted that FIG. 6 shows temporal changes in the amount of free glucose and the amount of xylose in the fermenter.

Rate of conversion of glucan into ethanol (%)=100×(amount of ethanol produced by S. cerevisiae)/(0.511×glucan amount in untreated rice straw starting material/0.9) [Eq. 8]

Rate of conversion of xylan into ethanol (%)=100×(amount of ethanol produced by P. stipitis)/(0.511×xylan amount in untreated rice straw starting material/0.88) [Eq. 9]

Total conversion rate into ethanol (%)=100×(ethanol amount in fermenter)/{0.511×(glucan amount in untreated rice straw starting material/0.9+xylan amount in untreated rice straw starting material/0.88)} [Eq. 10]

The results reveal that production of ethanol derived from glucan became gradual 16 hours after the start of culture or later, and the rate (%) of conversion of glucan into ethanol by S. cerevisiae was found to be 73% until 22 hours after the start. Meanwhile, glucose in the culture container was no longer detected immediate after culture of S. cerevisiae or later.

It should be noted that, as shown in Example 4, in the case where the saccharification reaction was conducted after the treatment with calcium hydroxide (4%, 120° C., 1 hour) under the same conditions as in this example, the glucan saccharification rate was found to be 77%. This suggests that calcium carbonate generated in the step of neutralization with carbon dioxide has no effect on the enzymatic reactions and growth of the yeast in the simultaneous saccharification and fermentation.

On the other hand, the concentration of xylose continuously increased in the fermenter before inoculation of P. stipitis (until 22 hours after the start of the simultaneous saccharification and fermentation), but started to decrease after inoculation of P. stipitis, and xylose was no longer detected 67 hours after the start of the simultaneous saccharification and fermentation or later. If ethanol produced in a period from inoculation of P. stipitis to 55 hours after the start of the simultaneous saccharification and fermentation was defined as ethanol derived from xylan, the rate of conversion of xylan into ethanol was found to be 44.8%.

In addition, the ‘total alcohol conversion rate’ in the period from the start of the simultaneous saccharification and fermentation to 55 hours after the start of simultaneous saccharification and fermentation was found to be 66%.

### Example 12

### Collection of Calcium Hydroxide from Fermentation Residue

The fermentation residue (rice straw) after the simultaneous saccharification and fermentation in Example 11 was collected by centrifugation (80,000 g, 20 minutes). After collection, the residue was dried at 65° C. for 2 days, and the dry weight was measured.

The dried fermentation residue was weighed in an amount of 1 g, placed in a crucible, and treated in a muffle furnace (FB-1314M, Barnsteadlthermolyne) at 1,000° C. for 1 hour. One hour later, the crucible was cooled at room temperature, and amounts of calcium oxide (CaO) derived from calcium hydroxide and ash derived from the rice straw were measured. After measurement, the combustion product was added to 100 mL of ultrapure water, and the mixture was stirred, followed by neutralization titration to pH 7 with 5 M hydrochloric acid and 0.1 M hydrochloric acid while measuring the pH. More specifically, hydrochloric acid necessary for neutralization of calcium hydroxide produced by a reaction of calcium oxide in the combustion product with water was quantified and converted into an amount of calcium hydroxide to determine a yield of calcium hydroxide.

The results reveal that the dry weight of the fermentation residue was 42.8 g. The weight decreased by 49% after combustion at 1,000° C. (dried residue: 1 g), and hence it was considered that calcium oxide and ash of rice straw accounted for 51% of the dry weight. Further, the amount of hydrochloric acid required for the neutralization reaction of the combustion product was 7.7 mmol, and hence the amount of calcium hydroxide collected in this step was calculated to be 3.85 mmol (0.285 g). This shows that 61.1% (12.2 g) of calcium hydroxide can be collected from calcium hydroxide (20 g) used in the alkali treatment.

### Example 13

### Collection of Phosphoric Acid from Fermentation Residue

Phosphoric acid (PO₄³⁻) in the combustion product collected in Example 12 was quantified by a modified molybdenum blue method. To 50 mg of the combustion product were added 1.2 ml of a 1 M/L sulfuric acid solution, and the mixture was subjected to ultrasonication for 5 minutes and further mixed using a vortex mixer for 5 minutes to extract phosphoric acid. The mixed solution was centrifuged, and the supernatant was used as a sample. As standard solutions, potassium dihydrogen phosphate solutions (0, 10, 25, and 50 ppm) were prepared and used.

After the sample or the standard solution had been mixed with a coloring reagent, the absorbance thereof at 880 nm was measured to calculate the concentration of phosphoric acid (PO₄³).

The results reveal that 1.6 g (corresponding to 7.2% of the combustion product) of phosphoric acid (PO₄³⁻) were able to be collected from the combustion product of a fermentation residue whose dry weight was 42.8 g.

### INDUSTRIAL APPLICABILITY

The present invention relates to development of an efficient technology for saccharifying a lignocellulosic biomass feedstock (including a lignocellulosic biomass feedstock containing readily degradable carbohydrates) and is expected to lead to development of a bioethanol production technology or development of a biorefinery technology.

In particular, the present invention is very important as an innovation in development of a domestic bioethanol production technology, which is an urgent issue in Japan.

1. A method of producing a slurry, the method comprising:
pulverizing an aerial part of a plant to obtain a lignocellulosic biomass feedstock;
combining the biomass feedstock, calcium hydroxide, and water to obtain a slurry;
treating the slurry in an alkali treatment; and
neutralizing the slurry by introduction of, pressurization with, or both introduction of and pressurization with carbon dioxide,
wherein the neutralizing provides a pH of the slurry of 5 to 7.

2. The method of claim 1, wherein a temperature of the alkali treatment is from 80 to 180° C. for from 10 minutes to 3 hours.

3. The method of claim 1, wherein a temperature of the alkali treatment is from 0° C. to 50° C. for 3 days or more.

4. The method of claim 1, further comprising: grinding solid matter in the slurry before or after the neutralizing.

5. The method of claim 1, wherein the aerial part of the plant comprises a part of at least one plant selected from the group consisting of rice, wheat, barley, corn, sugarcane, sorghum, erianthus, a pasture plant, and a monocotyledonous weed.

6. The method of claim 1, wherein the aerial part of the plant is a non-edible part.

7. A method of enzymatic saccharification, comprising:
adding a saccharification enzyme for at least one substance selected from the group consisting of starch, β-(1→33), (1→4)-glucan; cellulose; xylan; and partial degradation products thereof to a slurry obtained by the method of claim 1; and
reacting the slurry in an enzymatic saccharification reaction optionally comprising introduction of, pressurization with, or both introduction of and pressurization with carbon dioxide to prevent an increase in pH.

8. A method for ethanol production, comprising:
adding a microorganism for ethanol fermentation to a slurry comprising a saccharification product obtained by the method of claim 7; and
fermenting the slurry in an ethanol fermentation, optionally comprising introduction of, pressurization with, or both introduction of and pressurization with carbon dioxide to prevent an increase in pH.

9. The method of claim 7, further comprising:
adding microorganisms for ethanol fermentation,
wherein reacting the slurry in an enzymatic saccharification reaction further comprises simultaneous ethanol fermentation.

10. The method of claim 8, wherein the microorganism is yeast.

11. A bioethanol obtained by the method of claim 8.

12. A method for collecting an inorganic material comprising a calcium salt, the method comprising:
conducting the method of claim 7 to obtain a saccharification product;
collecting the saccharification product;
separating the saccharification product in a solid-liquid separation of a residue by membrane filtration or centrifugation to obtain solid matter; and
combusting the solid matter to collect ash.

13. A method for collecting an inorganic material comprising a calcium salt, the method comprising:
conducting the method of claim 8, to obtain ethanol and a residue;
collecting the ethanol;
conducting solid-liquid separation of the residue by membrane filtration or centrifugation to obtain solid matter; and
combusting the resultant solid matter to collect ash.

14. The method of claim 12, wherein the inorganic material comprises a phosphate.

15. An inorganic material, comprising a calcium salt, obtained by the method of claim 12.

16. The method of claim 9, wherein the microorganism for ethanol fermentation is yeast.

17. A method for collecting an inorganic material comprising a calcium salt, the method comprising:
conducting the method of claim 9, to obtain ethanol and a residue;
collecting the ethanol;
conducting solid-liquid separation of the residue by membrane filtration or centrifugation to obtain solid matter; and
combusting the resultant solid matter to collect ash.

18. The method of claim 13, wherein the inorganic material comprises a phosphate.

19. The method of claim 17, wherein the inorganic material comprises a phosphate.

20. The method of claim 7, wherein two or more saccharification enzymes are added to the slurry.
